(19) United States
(12) Patent Application Publication
(10) Pub. No.: US 2006/0115454 A1
(43) Pub. Date: Jun. 1, 2006

(54) Therapeutic RNAi agents for treating restenosis

(76) Inventors: Linda Couto, Pleasanton, CA (US); Sarah Brashears, Santa Clara, CA (US); Sara M. Cunningham, Lee's Summit, MO (US)

Correspondence Address: PATTERSON & SHERIDAN, L.L.P., 3040 POST OAK BOULEVARD, SUITE 1500, HOUSTON, TX, 77056, US

(21) Appl. No.: 11/251,076

(22) Filed: Oct. 14, 2005

Related U.S. Application Data
(60) Provisional application No. 60/619,103, filed on Oct. 15, 2004.
(60) Provisional application No. 60/627,400, filed on Nov. 12, 2004.

(51) Int. Cl. A61K 48/00; A61F 2/00

(52) U.S. Cl. 424/93.2; 514/44; 424/423

(57) ABSTRACT

The present invention provides compositions and methods suitable for delivering RNAi agents against genetic targets in vascular and adjacent tissue in vivo so as to treat restenosis.

### CROSS-REFERENCE TO RELATED APPLICATIONS

This application claims benefit of U.S. Provisional Patent Application Ser. No. 60/619,103, filed Oct. 15, 2004 and U.S. Provisional Patent Application Ser. No. 60/627,400, filed Nov. 12, 2004, which are herein incorporated by reference.

### FIELD OF THE INVENTION

The field of the invention is the treatment of restenosis. The present invention provides compositions and methods for the treatment of restenosis. The preferred embodiments of the compositions and methods relate to double stranded RNA used to interfere with the expression of genes involved in restenosis.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Utilization of double-stranded RNA to inhibit gene expression in a sequence-specific manner has revolutionized the drug discovery industry. In mammals, RNA interference, or RNAi, is mediated by 15- to 49-nucleotide long, double-stranded RNA molecules referred to as small interfering RNAs (RNAi agents). RNAi agents can be synthesized chemically or enzymatically outside of cells and subsequently delivered to cells (see, e.g., Fire, et al., Nature 391:806-11 (1998)); Tuschl, et al, Genes and Dev., 13:3191-97 (1999); and Elbashir, et al, Nature, 411:494-498 (2001); or can be expressed in vivo by an appropriate vector in cells (see, e.g., U.S. Pat. No. 6,573,099).

In vivo delivery of unmodified RNAi agents as an effective therapeutic for use in humans faces a number of technical hurdles. First, due to cellular and serum nucleases, the half life of RNA injected in vivo is only about 70 seconds (see, e.g., Kurreck, Eur. J. Bioch. 270:1628-44 (2003)). Efforts have been made to increase stability of injected RNA by the use of chemical modifications; however, there are several instances where chemical alterations led to increased cytotoxic effects. In one specific example, cells were intolerant to doses of an RNAi duplex in which every second phosphate was replaced by phosphorothioate (Harborth, et al, Antisense Nucleic Acid Drug Rev. 13(2): 83-105 (2003)). Still efforts continue to find ways to delivery unmodified or modified RNAi agents so as to provide tissue-specific delivery, as well as deliver the RNAi agents in amounts sufficient to elicit a therapeutic response but that are not toxic.

Other options being explored for RNAi delivery include the use of viral-based and non-viral based vector systems that can infect or otherwise transfect target cells, and deliver and express RNAi molecules in situ. Often, small RNAs are transcribed as short hairpin RNA (shRNA) precursors from a viral or non-viral vector backbone. Once transcribed, the shRNA are processed by the enzyme Dicer into the appropriate active RNAi agents. Viral-based delivery approaches attempt to exploit the targeting properties of viruses to generate tissue specificity and once appropriately targeted, rely upon the endogenous cellular machinery to generate sufficient levels of the RNAi agents to achieve a therapeutically effective dose.

One useful application of RNAi therapeutics is to treat restenosis. In general, coronary arteries are often subject to attack by disease processes, most commonly by atherosclerosis. In atherosclerosis, the coronary vessels become lined with lesions known as plaques. The development of plaques leads to a decrease in vessel cross-sectional area and a concomitant compromise in blood flow through the vessel. The reduction in blood flow to the coronary muscle can result in clinical angina, unstable angina or myocardial infarction and death.

Historically, the treatment of advanced atherosclerotic vascular disease involved cardio-thoracic surgery in the form of coronary artery bypass grafting (CABG). Another commonly used method for restoring blood flow to occluded vasculature is percutaneous coronary angioplasty.

The treatment of intravascular diseases by angioplasty is relatively non-invasive. Techniques, such as percutaneous transluminal angioplasty (PTA) and percutaneous transluminal coronary angioplasty (PTCA) typically involve use of a guide wire. A typical balloon catheter has an elongate shaft with a balloon attached to its distal end and a manifold attached to the proximal end. In use, the balloon catheter is advanced over the guide wire such that the balloon is positioned adjacent a restriction, in a diseased vessel. The balloon is then inflated and the restriction in the vessel is dilated.

Vascular restrictions that have been dilated do not always remain open. In up to 50% of the cases, a new restriction in the lumen of the vascular structure appears over a period of months. The newly formed restriction, or “restenosis,” arises due to the onset and maintenance of intimal hyperplasia at the site of insult. Restenosis and intimal hyperplasia following a procedure on a vascular structure is discussed in the following publications, see, for example Khanolkar, Indian Heart J. 48:281-282 (1996); Ghannem et al., Ann. Cardiol. Angeiol. 45:287-290 (1996); Macander et al., Cathet. Cardiovasc. Diagn. 32:125-131; Strauss et al., J. Am. Coil. Cardiol. 20:1465-1473 (1992); Bowerman et al., Cathet. Cardiovasc. Diagn. 24:248-251 (1991); Moris et al., Am. Heart. J. 131:834-836 (1996); Schomig et al., J. Am. Coll. Cardiol. 23:1053-1060 (1994); Gordon et al., J. Am. Coli. Cardiol. 21:1166-1174; and Baim et al., Am. J. Cardiol. 71:364-366 (1993).

Intimal hyperplasia also arises in conjunction with vascular reconstructive surgery. Vascular reconstructive surgery involves removing or reinforcing an area of diseased vasculature. Following removal of the diseased portion of the vessel, a prosthetic device, such as an endovascular stent graft or prosthetic graft is implanted at the site of removal. The graft is typically a segment of autologous or heterologous vasculature or, alternatively, it is a synthetic device fabricated from a polymeric material. Stent grafts are generally fabricated from metals, polymers and combinations of these materials. Similar to the situation with angioplasty, intimal hyperplasia also causes failure of implanted prosthetics in vascular reconstructive surgery. Thus, a method to reduce the failure rate for angioplasty and vascular reconstructive surgery by preventing or reducing intimal hyperplasia is an avidly sought goal.

Intimal hyperplasia is the result of a complex series of biological processes initiated by vascular injury followed by platelet aggregation and thrombus formation with a final pathway of smooth muscle cell migration and proliferation and extracellular matrix deposition. Platelets adhere and aggregate at the site of injury and release biologically active substances, the most important of which are platelet-derived growth factors (Scharf, et al, Blut 55:1131-1144 (1987)). It has been postulated that intimal hyperplasia production is driven by two principal mechanisms; platelet activation with the release of platelet-derived growth factors, and activation of the coagulation cascade with thrombus formation, which also results in the release of biologically active substances, which can contribute to smooth muscle cell proliferation (Chervu, et al, Surg. Gvnecol Obstet. 171:433-447, 1990)).

Attempts to prevent the onset, or to mitigate the effects, of intimal hyperplasia have included, for example, drug therapy with anti hyperplastic agents, such as antiplatelet agents (e.g. aspirin, arachidonic acid, prostacyclin), antibodies to platelet-derived growth factors, and antithrombotic agents (e.g. heparin, low molecular weight heparins) (see, Ragosta et al, Circulation 89: 11262-127 (1994)). Clinical trials using antihyperplastic agents, however, have shown little effect on the rate of restenosis (Schwartz, et al., N. Engl J. Med. 318:1714-1719, (1988); Meier, Eur. Heart J. 10 (suppl G):64-68 (1989)). In both angioplasty and vascular reconstructive surgery, drug infusion near the site of stenosis has been proposed as a means to inhibit restenosis. For example, U.S. Pat. No. 5,558,642 to Schweich, et al. describes drug delivery devices and methods for delivering pharmacological agents to vessel walls in conjunction with angioplasty.

Thus, there is a need in the art to develop stable, effective RNAi therapeutics for the treatment of restenosis. The present invention satisfies this need in the art.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is directed to genetic constructs and methods for delivering RNAi agents to vascular tissue to treat restenosis. In one aspect, the present invention provides innovative nucleic acid molecules comprising one or more RNAi agents for modifying target gene expression in vascular and/or surrounding tissues to treat restenosis. A further aspect of the present invention provides a genetic construct, that is capable of modifying the expression of one or more target genes where the genetic construct comprises one or more RNAi agents that are substantially identical to a sequence selected from (a) a nucleotide sequence from at least a region of the one or more target genes; (b) a derivative of (a); or (c) a sequence complementary to (a) or (b).

Thus, in a first aspect, the present invention provides a method of preventing or reducing restenosis, including blood clotting and/or intimal hyperplasia, at a site of insult to a blood vessel in a subject. The method comprises contacting an interior or exterior surface of the blood vessel with a therapeutic device comprising an RNAi agent, a carrier or coating and, in some embodiments, a delivery vehicle. In some embodiments, the carrier or coating is the delivery vehicle. The delivery vehicle may be a stent or stent graft inserted into the vessel, a drug delivery matrix wrapped around the vessel or a flowable liquid or semi-liquid material that is flowed or deposited onto the exterior surface of the blood vessel to which it will substantially adhere. The therapeutic device comprises one or more restenosis-preventing agents that are released from the therapeutic device in, preferably, a time dependent manner and in an amount effective to prevent or reduce restenosis.

Other objects and advantages of the present invention will be apparent from the detailed description that follows.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a simplified block diagram of one embodiment of a method for delivering RNAi agents to treat restenosis in vascular tissue according to the present invention.

FIGS. 2A and 2B show two embodiments of single-expression RNAi cassettes, and FIGS. 2C and 2D show two embodiments of multiple-expression RNAi cassettes.

FIGS. 3A and 3B show two embodiments of multiple expression cassettes that code for RNAi agents initially expressed as shRNA precursors, and FIGS. 3C and 3D show two embodiments of multiple expression cassettes that code for RNAi agents that are not expressed as shRNA precursors.

FIGS. 4A and 4B show alternative methods for producing viral particles for delivery of ddRNAi agents to vascular tissue.

### DETAILED DESCRIPTION

Before the present compositions and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention, which will be limited only by appended claims.

As used herein, the singular forms “a,” “an,” and “the” include plural referents unless the context clearly dictates otherwise. Thus, for example, reference to “a factor” refers to one or mixtures of factors, and reference to “the method of production” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference, without limitation, for the purpose of describing and disclosing devices, formulations and methodologies which are described in the publication and which might be used in connection with the presently described invention.

In the following description, numerous specific details are set forth to provide a more thorough understanding of the present invention. However, it will be apparent to one skilled in the art that the present invention may be practiced without one or more of these specific details. In other instances, well-known features and procedures well known to those skilled in the art have not been described in order to avoid obscuring the invention.

The present invention is directed to innovative, robust genetic compositions and methods to treat restenosis. The compositions and methods provide stable, lasting inhibition of a target gene.

Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

A “vector” is a replicon, such as plasmid, phage, viral construct or cosmid, to which another DNA segment may be attached. Vectors are used to transduce and express the DNA segment in cells.

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear of nucleolar RNAs or any kind of RNA transcribed by any class of any RNA polymerase.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA mayor may not be integrated (covalently linked) into the genome of the cell. With respect to eukaryotic cells, a stably transformed cell is one in which the transforming DNA has become integrated into a host cell chromosome or is maintained extra-chromosomally so that the transforming DNA is inherited by daughter cells during cell replication or is a non-replicating, differentiated cell in which a persistent episome is present.

The term “RNA interference” or “RNAi” refers generally to a process in which a double-stranded RNA molecule changes the expression of a nucleic acid sequence with which the double-stranded or short hairpin RNA molecule shares substantial or total homology. The term or “RNAi agent” refers to an RNA sequence that elicits RNAi; and the term “ddRNAi agent” refers to an RNAi agent that is transcribed from a vector. The terms “short hairpin RNA” or “shRNA” refer to an RNA structure having a duplex region and a loop region. In some embodiments of the present invention, ddRNAi agents are expressed initially as shRNAs. The term “RNAi expression cassette” refers to a cassette according to embodiments of the present invention having at least one [promoter-RNAi agent-terminator] unit. The term “multiple promoter RNAi expression cassette” refers to an RNAi expression cassette comprising two or more [promoter-RNAi agent-terminator] units. The terms “RNAi expression construct” or “RNAi expression vector” refer to vectors containing an RNAi expression cassette.

“Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

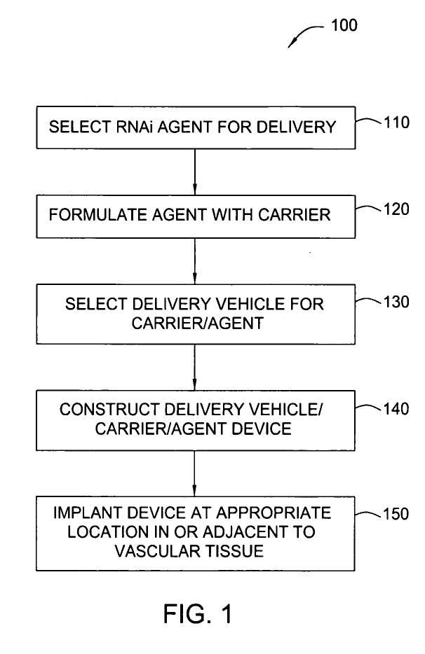

FIG. 1 is a simplified flow chart showing the steps of a method according to one embodiment of the present invention in which an RNAi agent according to the present invention may be used. Method 100 includes a step 110 in which an RNAi agent targeting a gene involved in restenosis is selected. Next, in step 120, the RNAi agent is formulated with an appropriate carrier or coating. A delivery vehicle, such as a stent, stent graft, matrix or flowable matrix is then selected at step 130, and, at step 140, the RNAi agent/carrier/delivery vehicle (collectively the therapeutic device) is constructed. Finally, at step 150, the therapeutic device is delivered to or implanted at an appropriate location in or adjacent to the vascular tissue in need of treatment.

RNAi agents according to the present invention can be generated synthetically or enzymatically by a number of different protocols known to those skilled in the art and purified using standard recombinant DNA techniques as described in, for example, Sambrook, et al., Molecular Cloning; A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research.

RNAi agents may comprise either siRNAs (synthetic RNAs) or DNA-directed RNAs (ddRNAs). siRNAs may be manufactured by methods known in the art such as by typical oligonucleotide synthesis, and often will incorporate chemical modifications to increase half life and/or efficacy of the siRNA agent, and/or to allow for a more robust delivery formulation. Many modifications of oligonucleotides are known in the art. For example, U.S. Pat. No. 6,620,805 discloses an oligonucleotide that is combined with a macrocycle having a net positive charge such as a porphyrin; U.S. Pat. No. 6,673,611 discloses various formulas; U.S. Publ. Nos. 2004/0171570, 2004/0171032, and 2004/0171031 disclose oligomers that include a modification comprising a polycyclic sugar surrogate; such as a cyclobutyl nucleoside, cyclopentyl nucleoside, proline nucleoside, cyclohexene nucleoside, hexose nucleoside or a cyclohexane nucleoside; and oligomers that include a non-phosphorous-containing internucleoside linkage; U.S. Publ. No. 2004/0171579 discloses a modified oligonucleotide where the modification is a 2′ substituent group on a sugar moiety that is not H or OH; U.S. Pub/. No. 2004/0171030 discloses a modified base for binding to a cytosine, uracil, or thymine base in the opposite strand comprising a boronated C and U or T modified binding base having a boron-containing substituent selected from the group consisting of —BH₂CN,— BH3, and —BH₂COOR, wherein R is C1 to C18 alkyl; U.S. Publ. No. 2004/0161844 discloses oligonucleotides having phosphoramidate internucleoside linkages such as a 3′aminophosphoramidate, aminoalkylphosphoramidate, or aminoalkylphosphorthioamidate internucleoside linkage; U.S. Publ. No. 2004/0161844 discloses yet other modified sugar and/or backbone modifications, where in some embodiments, the modification is a peptide nucleic acid, a peptide nucleic acid mimic, a morpholino nucleic acid, hexose sugar with an amide linkage, cyclohexenyl nucleic acid (CeNA), or an acyclic backbone moiety; U.S. Publ. No. 2004/0161777 discloses oligonucleotides with a 3′ terminal cap group; U.S. Publ. No. 2004/0147470 discloses oligomeric compounds that include one or more cross-linkages that improve nuclease resistance or modify or enhance the pharmacokinetic and phamacodynamic properties of the oligomeric compound where such cross-linkages comprise a disulfide, amide, amine, oxime, oxyamine, oxyimine, morpholino, thioether, urea, thiourea, or sulfonamide moiety; U.S. Publ. No. 2004147023 discloses a gapmer comprising two terminal RNA segments having nucleotides of a first type and an internal RNA segment having nucleotides of a second type where nucleotides of said first type independently include at least one sugar substituent where the sugar substituent comprises a halogen, amino, trifluoroalkyl, trifluoroalkoxy, azido, aminooxy, alkyl, alkenyl, alkynyl, O- S-, or N(R*)-alkyl; O-, S-, or N(R*)-alkenyl; O-, S- or N(R*)-alkynyl; O-, S- or N-aryl, O-, S-, or N(R*)-aralkyl group; where the alkyl, alkenyl, alkynyl, aryl or aralkyl may be a substituted or unsubstituted alkyl, alkenyl, alkynyl, aralkyl; and where, if substituted, the substitution is an alkoxy, thioalkoxy, phthalimido, halogen, amino, keto, carboxyl, nitro, nitroso, cyano, trifluoromethyl, trifluoromethoxy, imidazole, azido, hydrazino, aminooxy, isocyanato, sulfoxide, sulfone, disulfide, silyl, heterocycle, or carbocycle group, or an intercalator, reporter group, conjugate, polyamine, polyamide, polyalkylene glycol, or a polyether of the formula (—O-alkyl)ₘ, where m is 1 to about 10; and R* is hydrogen, or a protecting group; or U.S. Publ. No. 2004/0147022 disclosing an oligonucleotide with a modified sugar and/or backbone modification, such as a 2′-OCH₃ substituent group on a sugar moiety.

Alternatively, DNA-directed RNAi (ddRNAi) agents may be employed. ddRNAi agents comprise an expression cassette or ddRNAi expression cassette, most often comprising at least one promoter, at least one ddRNAi sequence and at least one terminator in a viral or non-viral vector backbone.

In one preferred embodiment, the ddRNAi expression cassette comprises a nucleic acid molecule comprising the general structure (I): 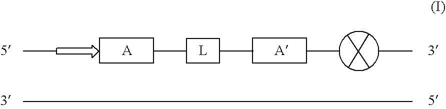
wherein: [symbol]
represents a promoter sequence; [symbol]
represents a ddRNAi targeting sequence comprising at least 10 nucleotides, wherein said sequence is at least 70% identical to a target sequence or part thereof; [symbol]
represents a sequence of 10 to 30 nucleotides wherein at least 10 contiguous nucleotides of A′ comprise a reverse complement of the nucleotide sequence represented by A; [symbol]
represents a “loop” encoding structure comprising a sequence of 5 to 20 non-self-complementary nucleotides; and [symbol]
represents a terminator sequence.

The ddRNAi agent generated by the expression of the ddRNAi expression cassette represented by general structure (I) comprises a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length. Accordingly, in a preferred embodiment, L in general structure (I) comprises 5, 6, 7, 8 or 9 non-self-complementary nucleotides.

In another embodiment, the ddRNAi expression cassette comprises a nucleic acid molecule of the general structure (II): 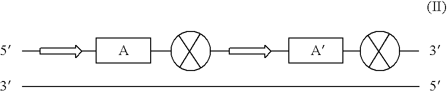
wherein: [symbol]
represents a promoter sequence; [symbol]
represents a ddRNAi targeting sequence comprising at least 10 nucleotides, wherein said sequence is at least 70% identical to a target sequence or part thereof; [symbol]
represents a sequence of 10 to 30 nucleotides wherein at least 10 contiguous nucleotides of A′ comprise a reverse complement of the nucleotide sequence represented by A; and [symbol]
represents a terminator sequence.

In yet another embodiment, the ddRNAi expression cassette comprises a nucleic acid molecule of the general structure (III): 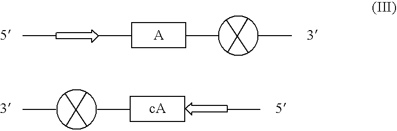
wherein: [symbol]
represents a promoter sequence; [symbol]
represents a ddRNAi targeting sequence comprising at least 10 nucleotides, wherein said sequence is at least 70% identical to a target sequence or part thereof; [symbol]
represents a nucleic acid sequence complementary to A; and [symbol]
represents a terminator sequence.

In yet another preferred embodiment, the ddRNAi expression cassette comprises a nucleic acid molecule of the general structure (IV): 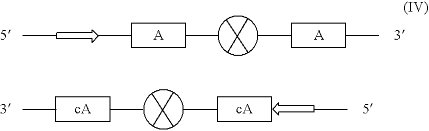
wherein: [symbol]
represents a promoter sequence; [symbol]
represents a ddRNAi targeting sequence comprising at least 10 nucleotides, wherein said sequence is at least 70% identical to a PAT sequence or part thereof;
[symbol] represents a nucleic acid sequence complementary to A; and represents a terminator sequence.

Although the ddRNAi expression cassettes represented by general structures (I), (II), (III) and (IV) represent preferred embodiments of the invention, the present invention is in no way limited to these particular general structures. As would be evident to one of skill in the art, the above structures may be modified while retaining functionality. For example, the elements of the cassettes may be separated by one or more nucleotide residues. Furthermore, elements which are present on complementary strands, such as the terminator and promoter elements shown in structures (III) and (IV) may overlap or may be discreet. For example, the terminator elements shown in structure (III) may occur within the complementary strand of the promoter element or may be upstream or downstream of this region. Other modifications which would be evident to one of skill in the art and which do not materially effect the functioning of the cassette in encoding a dsRNA stucture may also be made and such modified cassettes are within the scope of the present invention.

In addition, the ddRNAi expression cassettes may be configured where multiple cloning sites and/or unique restriction sites are located strategically, such that the promoter, ddRNAi agents and terminator elements are easily removed or replaced. The RNAi expression cassettes may be assembled from smaller oligonucleotide components using strategically located restriction sites and/or complementary sticky ends. The base vector for one approach according to embodiments of the present invention consists of plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into, e.g. an AVV backbone using two unique enzyme sites (the same or different ones) that flank the single-:, double- or multiple-expression cassette insert.

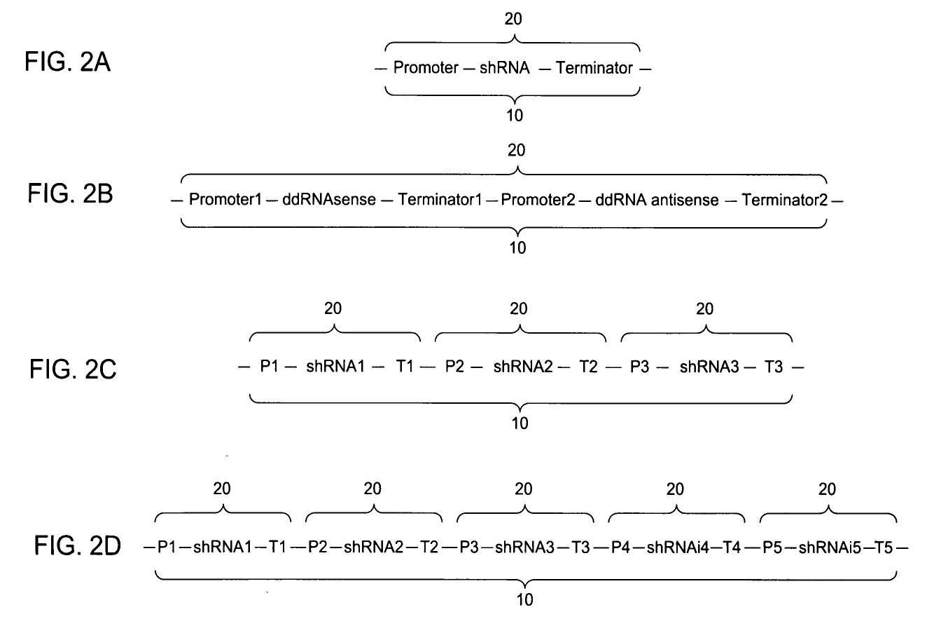

FIGS. 2A and 2B are simplified schematics of single-promoter RNAi expression cassettes according to embodiments' of the present invention. FIG. 2A shows an embodiment of a single RNAi expression cassette (10) comprising one promoter/RNAi/terminator component (shown at 20), where the ddRNAi agent is expressed initially as a short hairpin (shRNA). FIG. 2B shows an embodiment of a single. RNAi expression cassette (10) with one promoter/RNAi/terminator component (shown at 20), where the sense and antisense components of the ddRNAi agent are expressed separately from different promoters.

FIGS. 2C and 2D are simplified schematics of multiple-promoter RNAi expression cassettes according to embodiments of the present invention. FIG. 2C shows an embodiment of a multiple-promoter RNAi expression cassette (10) comprising three promoter/RNAi/terminator components (shown at 20), and FIG. 2D shows an embodiment of a multiple-promoter expression cassette (10) with five promoter/RNAilterminator components (shown at 20). P1, P2, P3, P4 and P5 represent promoter elements. RNAi1, RNAi2, RNAi3, RNAi4 and RNAi5 represent sequences for five different ddRNAi agents. T1, T2, T3, T4, and T5 represent termination elements. The multiple-promoter RNAi expression cassettes according to the present invention may contain two or more promoter/RNAi/terminator components where the number of promoter/RNAi/terminator components included in any multiple-promoter RNAi expression cassette is limited by, e.g., packaging size of the delivery system chosen (for example, some viruses, such as AAV, have relatively strict size limitations); cell toxicity, and maximum effectiveness (i.e. when, for example, expression of four ddRNAi agents is as effective therapeutically as the expression of ten ddRNAi agents).

When employing a multiple promoter RNAi expression cassette, the two or more ddRNAi agents in the promoter/RNAi/terminator components comprising a cassette all have different sequences; that is RNAi1, RNAi2, RNAi3, RNAi4 and RNAi5 are all different from one another. However, the promoter elements in any cassette may be the same (that is, e.g., the sequence•of two or more of P1, P2, P3, P4 and P5 may be the same); all the promoters within any cassette may be different from one another; or there may be a combination of promoter elements represented only once and promoter elements represented two times or more within any cassette. Similarly, the termination elements in any cassette may be the same (that is, e.g., the sequence of two or more of T1, T2, T3, T4 and T5 may be the same, such as contiguous stretches of 4 or more T residues); all the termination elements within any cassette may be different from one another; or there may be a combination of termination elements represented only once and termination elements represented two times or more within any cassette. Preferably, the promoter elements and termination elements in each promoter/RNAi/terminator component comprising any cassette are all different to decrease the likelihood of DNA recombination events between components and/or cassettes. Further, in a preferred embodiment, the promoter element and termination element used in each promoter/RNAi/terminator component are matched to each other; that is, the promoter and terminator elements are taken from the same gene in which they occur naturally.

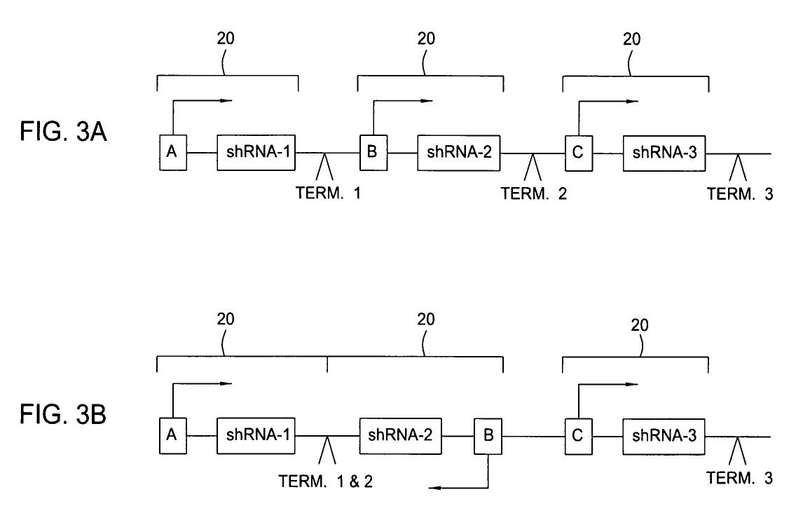

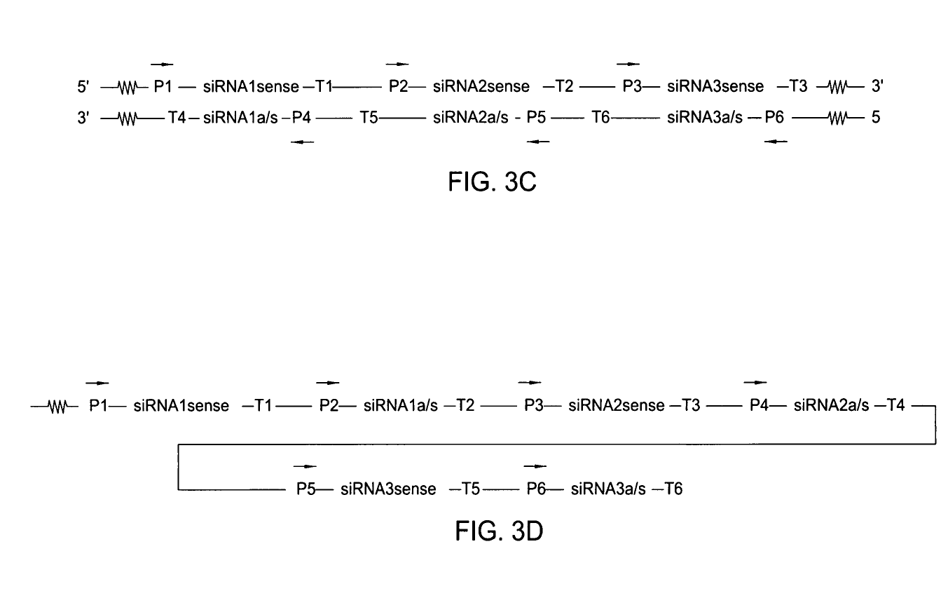

FIGS. 3A and 3B show multiple-promoter RNAi expression constructs comprising alternative embodiments of multiple-promoter RNAi expression cassettes that express short shRNAs. shRNAs are short duplexes where the sense and antisense strands are linked by a hairpin loop. Once expressed, shRNAs are processed into RNAi agents. A, B and C represent three different promoter elements, and the arrows indicate the direction of transcription. Term1, Term2, and Term3 represent three different termination sequences, and shRNA-1, shRNA-2 and shRNA-3 represent three different shRNA sequences. The multiple-promoter RNAi expression cassettes in both embodiments extend from the box marked A to the Term3. FIG. 3A shows each of the three promoter/RNAi/terminator components (20) in the same orientation within the cassette, while FIG. 3B shows the promoter/RNAi/terminator components for shRNA-1 and shRNA-3 in one orientation, and the promoter/RNAi/terminator component for sh-RNA2 in the opposite orientation (i.e., transcription takes place on both strands of the cassette). Other variations may be used as well.

FIGS. 3C and 3D show multiple-promoter RNAi expression constructs comprising alternative embodiments of multiple-promoter RNAi expression cassettes that express RNAi agents without a hairpin loop. In both figures, P1, P2, P3, P4, P5 and P6 represent promoter elements (with arrows indicating the direction of transcription); and T1, T2, T3, T4, T5, and T6 represent termination elements. Also in both figures, RNAi1 sense and RNAi1 antisense (a/s) are complements, RNAi2 sense and RNAi2 a/s are complements, and RNAi3 sense and RNAi3 a/s are complements.

In the embodiment shown in FIG. 3C, all three RNAi sense sequences are transcribed from one strand (via P1, P2 and P3), while the three RNAi a/s sequences are transcribed from the complementary strand (via P4, P5, P6). In this particular embodiment, the termination element of RNAi1 a/s (T4) falls between promoter P1 and the RNAi 1 sense sequence; while the termination element of RNAi1 sense (T1) falls between the RNAi 1 a/s sequence and its promoter, P4. This motif is repeated such that if the top strand shown in FIG. 3C is designated the (+) strand and the bottom strand is designated the (−) strand, the elements encountered moving from left to right would be P1 (+), T4(−), RNAi1 (sense and a/s), T1 (+), P4(−), P2(+), T5(−), RNAi2 (sense and a's), T2(+), P5(−), P3(+), T6(−), RNAi3 (sense and a/s), T3(+), and P6(−).

In an alternative embodiment shown in FIG. 3D, all RNAi sense and antisense sequences are transcribed from the same strand. One skilled in the art appreciates that any of the embodiments of the multiple-promoter RNAi expression cassettes shown in FIGS. 3A through 3D may be used for certain applications, as well as combinations or variations thereof.

In some embodiments, promoters of variable strength may be employed. For example, use of two or more strong promoters (such as a Pol III-type promoter) may tax the cell, by, e.g., depleting the pool of available nucleotides or other cellular components needed for transcription. In addition or alternatively, use of several strong promoters may cause a toxic level of expression of RNAi agents in the cell. Thus, in some embodiments one or more of the promoters in the multiple-promoter RNAi expression cassette may be weaker than other promoters in the cassette, or all promoters in the cassette may express RNAi agents at less than a maximum rate. Promoters also mayor may not be modified using molecular techniques, or otherwise, e.g., through regulation elements, to attain weaker levels of transcription.

Promoters useful in some embodiments of the present invention may be tissue-specific or cell-specific. The term “tissue specific” as it applies to a promoter refers to a promoter that is capable of directing selective expression of a nucleotide sequence of interest to a specific type of tissue (e.g., vascular tissue) in the relative absence of expression of the same nucleotide sequence of interest in a different type of tissue (e.g., muscle). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, e.g., Higashibata, et al, J. Bone Miner. Res. January 19(1):78-88 (2004); Hoggatt, et al, Circ. Res., December 91(12):1151-59 (2002); Sohal, et al, Circ. Res. July 89(1):20-25 (2001); and Zhang, et al, Genome Res. January 14(1):79-89 (2004)). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “constitutive” when made in reference to a promoter means that the promoter is capable of directing transcription of an operably linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe the RNAi agents preferably are constitutive promoters, such as the promoters for ubiquitin, CMV, β-actin, histone H4, EF-1alfa or pgk genes controlled by RNA polymerase II, or promoter elements controlled by RNA polymerase I. In other embodiments, a Pol II promoter such as CMV, SV40, U1, β-actin or a hybrid Pol II promoter is employed. In other embodiments, promoter elements controlled by RNA polymerase III are used, such as the U6 promoters (U6-1, U6-8, U6-9, e.g.), H1 promoter, 7SL promoter, the human Y promoters (hY1, hY3, hY 4 (see Maraia, et al., Nucleic Acids Res 22(15):3045-52 (1994)) and hY5 (see Maraia, et al., Nucleic Acids Res 24(18):3552-59 (1994)), the human MRP-7-2 promoter, Adenovirus VA1 promoter, human tRNA promoters, the 5s ribosomal RNA promoters, as well as functional hybrids and combinations of any of these promoters.

Alternatively in some embodiments it may be optimal to select promoters that allow for inducible expression of the RNAi agent. A number of systems for inducible expression using such promoters are known in the art, including but not limited to the tetracycline responsive system and the lac operator-repressor system (see WO 03/022052 A1; and US 2002/0162126 A1), the ecdyson regulated system, or promoters regulated by glucocorticoids, progestins, estrogen, RU-486, steroids, thyroid hormones, cyclic AMP, cytokines, the calciferol family of regulators, or the metallothionein promoter (regulated by inorganic metals).

One or more enhancers also may be present in the viral multiple-promoter RNAi expression construct to increase expression of the gene of interest. Enhancers appropriate for use in embodiments of the present invention include the Apo E HCR enhancer, the CMV enhancer that has been described recently (see, Xia, et ai, Nucleic Acids Res 31-17 (2003)), and other enhancers known to those skilled in the art.

The RNAi sequences encoded by the RNAi expression cassettes of the present invention result in the expression of small interfering RNAs that are short, double-stranded RNAs that are not toxic in normal mammalian cells. There is no particular limitation in the length of the ddRNAi agents of the present invention-as long as they do not show cellular toxicity. RNAis can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

The termini of a ddRNAi agent according to the present invention may be blunt or cohesive (overhanging) as long as the ddRNAi agent effectively silences the target gene. The cohesive (overhanging) end structure is not limited only to a 3′ overhang, but a 5′ overhanging structure may be included as long as the resulting ddRNAi agent is capable of inducing the RNAi effect. In addition, the number of overhanging nucleotides may be any number as long as the resulting ddRNAi agent is capable of inducing the RNAi effect. For example, if present, the overhang may consist of 1 to 8 nucleotides, preferably it consists of 2 to 4 nucleotides.

The ddRNAi agent utilized in the present invention may have a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length.

The nucleic acid sequences that are targets for the RNAi expression cassettes of the present invention include genes that are involved in restenosis in general, including but not limited to blood clotting (the coagulation cascade) and smooth muscle or endothelial proliferation (intimal hyplasia) (collectively, “target genes”). The sequences for the RNAi agent or agents are selected based upon the genetic sequence of the target gene sequence(s); and preferably are based on regions of the target gene sequences that are conserved. Methods of alignment of sequences for comparison and RNAi sequence selection are well-known in the art. The determination of percent identity between two or more sequences can be accomplished using a mathematical algorithm. Preferred, non-limiting examples of such mathematical algorithms are the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (http://www.ncbi.nlm.nih.gov/).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Typically, inhibition of target sequences by RNAi requires a high degree of sequence homology between the target sequence and the sense strand of the RNAi molecules. In some embodiments, such homology is higher than about 70%, and may be higher than about 75%. Preferably, homology is higher than about 80%, and is higher than 85% or even 90%. More preferably, sequence homology between the target sequence and the sense strand of the RNAi is higher than about 90%, 91%, 92%, 93%, 94%, 95%, 96%, 97%, 98% or 99%.

In addition to selecting the RNAi sequences based on conserved regions of a target gene, selection of the RNAi sequences may be based on other factors. Despite a number of attempts to devise selection criteria for identifying sequences that will be effective in RNAi based on features of the desired target sequence (e.g., percent GC content, position from the translation start codon, or sequence similarities based on an in silico sequence database search for homologs of the proposed RNAi, thermodynamic pairing criteria), it is presently not possible to predict with much degree of confidence which of the myriad possible candidate RNAi sequences correspond to target gene, in fact, elicit an optimal RNA silencing response. Instead, individual specific candidate RNAi polynucleotide sequences typically are generated and tested to determine whether interference with expression of a desired target can be elicited.

As stated, the ddRNAi agent coding regions of RNAi expression cassette are operatively linked to terminator elements. In one embodiment, the terminators comprise stretches of four or more thymidine residues. In embodiments where multiple promoter cassettes are used, the terminator elements used all may be different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the RNAi expression cassettes may be configured where multiple cloning sites and/or unique restriction sites are located strategically, such that the promoter, ddRNAi agents and terminator elements are easily removed or replaced. The RNAi expression cassettes may be assembled from smaller oligonucleotide components using strategically located restriction sites and/or complementary sticky ends. The base vector for one approach according to embodiments of the present invention consists of plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into, e.g. an AAV backbone using two unique enzyme sites (the same or different ones) that flank the single-, double- or multiple-expression cassette insert.

When using a ddRNAi agent, the RNAi expression cassette is ligated into a delivery vector. The constructs into which the RNAi expression cassette is inserted and used for high efficiency transduction and expression of the ddRNAi agents in various cell types may be derived from viruses- and are compatible with viral delivery; alternatively, non-viral delivery method may be used. Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. If the construct is a viral construct, the construct preferably comprises, for example, sequences necessary to package the RNAi expression construct into viral particles and/or sequences that allow integration of the RNAi expression construct into the target cell genome. The viral construct also may contain genes that allow for replication and propagation of virus, though in other embodiments such genes will be supplied in trans. Additionally, the viral construct may contain genes or genetic sequences from the genome of any known organism incorporated in native form or modified. For example, a preferred viral construct may comprise sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the RNAi expression cassette, an internal ribosomal entry site (IRES) sequence can be included. Preferably, the additional genetic elements are operably linked with and controlled by an independent promoter/enhancer. In addition a suitable origin of replication for propagation of the construct in bacteria may be employed. The sequence of the origin of replication generally is separated from the ddRNAi agent and other genetic sequences that are to be expressed in the vascular tissue. Such origins of replication are known in the art and include the pUC, ColE1, 2-micron or SV40 origins of replication.

A viral delivery system based on any appropriate virus may be used to deliver the RNAi expression constructs of the present invention. In addition, hybrid viral systems may be of use. The choice of viral delivery system will depend on various parameters, such as efficiency of delivery into vascular tissue, transduction efficiency of the system, pathogenicity, immunological and toxicity concerns, and the like. It is clear that there is no single viral system that is suitable for all applications. When selecting a viral delivery system to use in the present invention, it is important to choose a system where RNAi expression construct-containing viral particles are preferably: 1) reproducibly and stably propagated; 2) able to be purified to high titers; and 3) able to mediate targeted delivery (delivery of the multiple-promoter RNAi expression construct to the vascular tissue without widespread dissemination).

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transducer a target cell by receptor-mediated binding and internalization, penetrating the nucleus in both non-dividing and dividing cells.

Once in the nucleus, the virus uncoats and the transgene is expressed from a number of different forms-the most persistent of which are circular monomers. MV will integrate into the genome of 1-5% of cells that are stably transduced (Nakai, et al., J. ViroL 76:11343-349 (2002). Expression of the transgene can be exceptionally stable and in one study with MV delivery of Factor IX, a dog model continues to express therapeutic levels of the protein 4.5 years after a single direct infusion with the virus. Because progeny virus is not produced from AAV infection in the absence of helper virus, the extent of transduction is restricted only to the initial cells that are infected with the virus. It is this feature that makes AAV a preferred gene therapy vector for the present invention. Furthermore, unlike retrovirus, adenovirus, and herpes simplex virus, AAV appears to lack human pathogenicity and toxicity (Kay, et al., Nature. 424: 251 (2003) and Thomas, et al., Nature Reviews, Genetics 4:346-58 (2003)).

Typically, the genome of AAV contains only two genes. The “rep” gene codes for at least four separate proteins utilized in DNA replication. The “cap” gene product is spliced differentially to generate the three proteins that comprise the capsid of the virus. When packaging the genome into nascent virus, only the Inverted Terminal Repeats (ITRs) are obligate sequences; rep and cap can be deleted from the genome and be replaced with heterologous sequences of choice. However, in order produce the proteins needed to replicate and package the AA V-based heterologous construct into nascent virion, the rep and cap proteins must be provided in trans. The helper functions normally provided by co-infection with the helper virus, such as adenovirus or herpesvirus mentioned above, also can be provided in trans in the form of one or more DNA expression plasmids. Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as RNAi.

The utility of AAV for RNAi applications was demonstrated in experiments where AA V was used to deliver shRNA in vitro to inhibit p53 and Caspase 8 expression (Tomar et al., OncoQene. 22: 5712-15 (2003)). Following cloning of the appropriate sequences into a gutted AAV-2 vector, infectious AAV virions were generated In HEK293 cells and used to infect HeLa S3 cells. A dose-dependent decrease of endogenous Caspase 8 and p53 levels was demonstrated. Boden et al. also used MV to deliver shRNA in vitro to inhibit HIV replication in tissue culture systems (Boden, et al., J. Virol. 77(21): 115231-35 (2003)) as assessed by p24 production in the spent media.

However, technical hurdles must be addressed when using AAV as a vehicle for RNAi expression constructs. For example, various percentages of the human population may possess neutralizing antibodies against certain AAV serotypes. However, since there are several AAV serotypes, some of which the percentage of individuals harboring neutralizing antibodies is vastly reduced, other serotypes can be used or pseudo-typing may be employed. There are at least eight different serotypes that have been characterized, with dozens of others, which have been isolated but have been less well described. Another limitation is that as a result of a possible immune response to AAV, AAV-based therapy may only be administered once; however, use of alternate, non-human derived serotypes may allow for repeat administrations. Administration route, serotype, and composition of the delivered genome all influence tissue specificity.

Another limitation in using unmodified AAV systems with the RNAi expression constructs is that transduction can be inefficient. Stable transduction in vivo may be limited to 5-10% of cells. However, different methods are known in the art to boost stable transduction levels. One approach is utilizing pseudotyping, where AAV-2 genomes are packaged using cap proteins derived from other serotypes. For example, by substituting the AAV-5 cap gene for its AAV-2 counterpart, Mingozzi et al. increased stable transduction to approximately 15% of hepatocytes (Mingozzi, et al., J. Virol. 76(20): 10497-502 (2002)). Thomas et al., transduced over 30% of mouse hepatocytes in vivo using the AAV8 capsid gene (Thomas, et al., J. Virol. in press). Grimm et al. (Blood. 2003-02-0495) exhaustively pseudotyped AAV-2 with AAV-1, AAV-3B, AAV-4, AAV-5, and AAV-6 for tissue culture studies. The highest levels of transgene expression were induced by virion which had been pseudotyped with MV-6; producing nearly 2000% higher transgene expression than AAV-2. Thus, the present invention contemplates use of a pseudotyped AAV virus to achieve high transduction levels, with a corresponding increase in the expression of the RNAi multiple-promoter expression constructs.

Another viral delivery system useful with the RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Reverse transcription of the retroviral RNA genome occurs in the cytoplasm. Unlike C-type “retroviruses, the lentiviral cDNA complexed with other viral factors-known as the pre-initiation complex-is able to translocate across the nuclear membrane and transduce non-dividing cells. A structural feature of the viral cDNA-a DNA flap-seems to contribute to efficient nuclear import. This flap is dependent on the integrity of a central polypurine tract (cPPT) that is located in the viral polymerase gene, so most lentiviral-derived vectors retain this sequence. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types.

A lentiviral-based construct used to express the ddRNAi agents preferably comprises sequences from the 5′ and 3′ LTRs of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the RNAi expression cassettes of the present invention to vascular tissue, including but not limited to gene-deleted adenovirus-transposon vectors that stably maintain virus-encoded transgenes in vivo through integration into host cells (see Yant, et al., Nature Biotech. 20:999-1004 (2002)); systems derived from Sindbis virus or Semliki forest virus (see Perri, et ai, J. Virol. 74(20):9802-07 (2002)); systems derived from Newcastle disease virus or Sendai Virus; or mini-circle DNA vectors devoid of bacterial DNA sequences (see Chen, et al., Molecular Therapy. 8(3):495-500 (2003)).

In addition, hybrid viral systems may be used to combine useful properties of two or more viral systems. For example, the site-specific integration machinery of wild-type AAV may be coupled with the efficient internalization and nuclear targeting properties of adenovirus. AAV in the presence of adenovirus or herpesvirus undergoes a productive replication cycle; however, in the absence of helper functions, the AAV genome integrates into a specific site on chromosome 19. Integration of the AAV genome requires expression of the AAV rep protein. As conventional rAAV vectors are deleted for all viral genes including rep, they are not able to specifically integrate into chromosome 19. However, this feature may be exploited in an appropriate hybrid system. In addition, non-viral genetic elements may be used to achieve desired properties in a viral delivery system, such as genetic elements that allow for site-specific recombination.

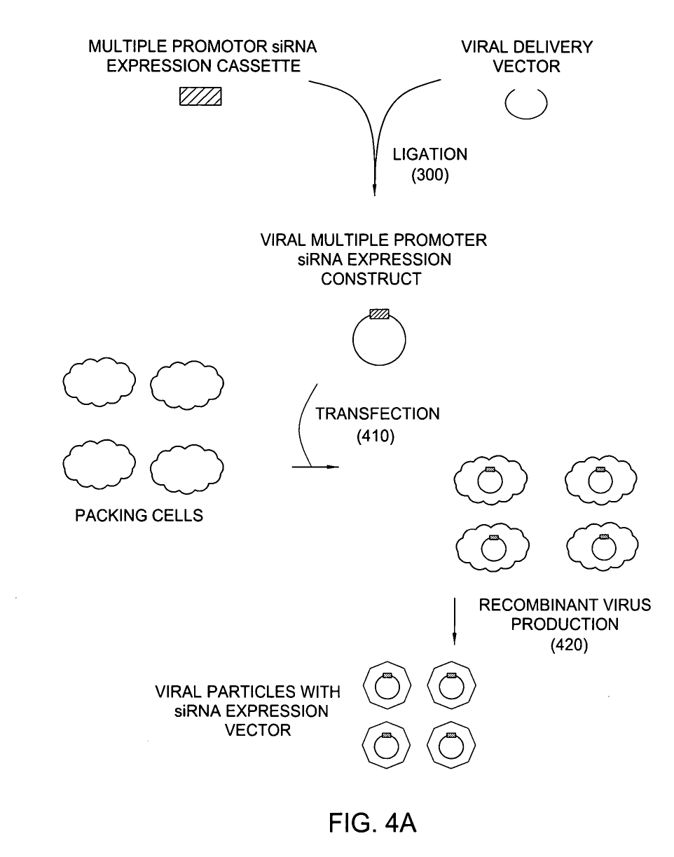

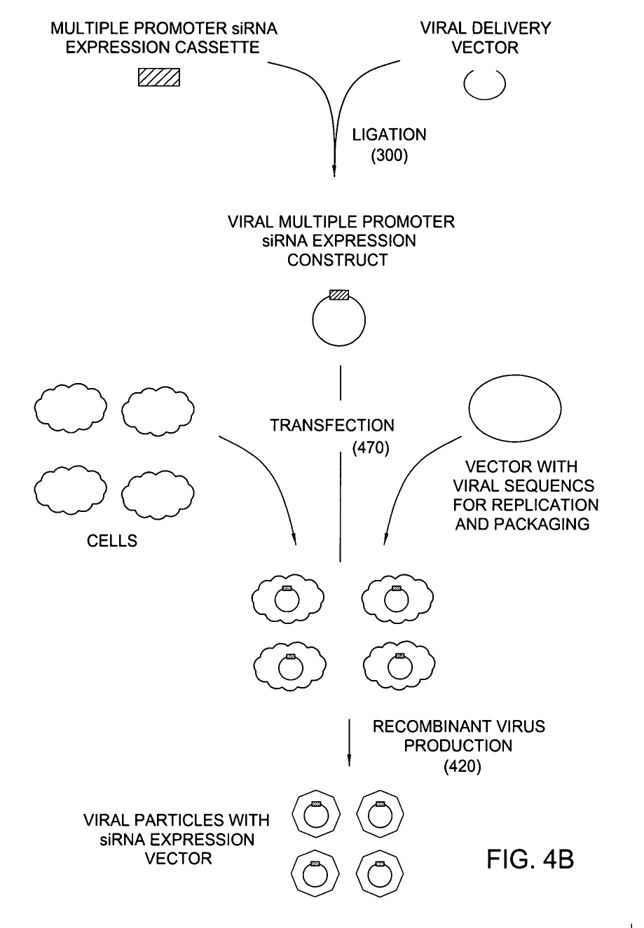

In step 130 of FIG. 1, the RNAi expression construct is packaged into viral particles. Any method known in the art may be used to produce infectious viral particles whose genome comprises a copy of the viral RNAi expression construct. FIGS. 4A and 4B show alternative methods for packaging the RNAi expression constructs of the present invention into viral particles for delivery. The method in FIG. 4A utilizes packaging cells that stably express in trans the viral proteins that are required for the incorporation of the viral RNAi expression construct into viral particles, as well as other sequences necessary or preferred for a particular viral delivery system (for example, sequences needed for replication, structural proteins and viral assembly) and either viral-derived or artificial ligands for tissue entry. In FIG. 4A, a RNAi expression cassette is ligated to a viral delivery vector (step 300), and the resulting viral RNAi expression construct is used to transfect packaging cells (step 410). The packaging cells then replicate viral sequences, express viral proteins and package the viral RNAi expression constructs into infectious viral particles (step 420). The packaging cell line may be any cell line that is capable of expressing viral proteins, including but not limited to 293, HeLa, A549, PerC6, 017, MDCK, BHK, bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the viral RNAi expression construct, and the other plasmid(s) comprises nucleic acids encoding the proteins necessary to allow the cells to produce functional virus (replication and packaging construct) as well as other helper functions. The method shown in FIG. 4B utilizes cells for packaging that do not stably express viral replication and packaging genes. In this case, the RNAi expression construct is ligated to the viral delivery vector (step 300) and then co-transfected with one or more vectors that express the viral sequences necessary for replication and production of infectious viral particles (step 430). The cells replicate viral sequences, express viral proteins and package the viral RNAi expression constructs into infectious viral particles (step 420).

The packaging cell line or replication and packaging construct may not express envelope gene products. In these embodiments, the gene encoding the envelope gene can be provided on a separate construct that is co-transfected with the viral RNAi expression construct. As the envelope protein is responsible, in part, for the host range of the viral particles, the viruses may be pseudotyped. As described supra, a “pseudotyped” virus is a viral particle having an envelope protein that is from a virus other than the virus from which the genome is derived. One with skill in the art can choose an appropriate pseudotype for the viral delivery system used and cell to be targeted. In addition to conferring a specific host range, a chosen pseudotype may permit the virus to be concentrated to a very high titer. Viruses alternatively can be pseudotyped with ecotropic envelope proteins that limit infection to a specific species (e.g., ecotropic envelopes allow infection of, e.g., murine cells only, where amphotropic envelopes allow infection of, e.g., both human and murine cells.) In addition, genetically-modified ligands can be used for cell-specific targeting, such as the asialoglycoprotein for hepatocytes, or transferrin for receptor-mediated binding.

After production in a packaging-cell line, the viral particles containing the RNAi expression cassettes are purified and quantified (titered). Purification strategies include density gradient centrifugation, or, preferably, column chromatographic methods.

Multiple-promoter RNAi expression cassettes used in certain embodiments of the present invention are particularly useful in treating restenosis because RNAi agents against multiple genes involved in restenosis can be targeted simultaneously. For example, one or more genes that regulate blood clotting and/or intimal hyperplasia can be repressed at the same time.

A variety of techniques are available and well known for delivery of nucleic acids into cells, for example liposome- or micelle-mediated transfection or transformation, transformation of cells with attenuated virus particles or bacterial cells, cell mating, transformation or transfection procedures known to those skilled in the art or microinjection. The RNAi agents, whether siRNAs or ddRNAs, are formulated with an appropriate carrier or coating, which may then be associated with a delivery vehicle such as a matrix or stent graft, ultimately forming a therapeutic device. The delivery vehicle may be the carrier itself that is a flowable liquid or gel that is applied to the exterior of a blood vessel, a synthetic or naturally-occurring matrix that is applied to the exterior of a blood vessel, or a vascular prosthesis that is implanted in a blood vessel. Preferred prostheses include, but are not limited to stents, grafts, valves or a combination thereof. Various chemical formulations for carrier/coatings, matrices, stents or stent grafts are presented in detail below.

One delivery vehicle appropriate for use in the present invention is a matrix. In general, a matrix is a scaffold comprising synthetic, semi-synthetic or naturally-occurring compounds that can be used as a delivery vehicle to deliver the RNAi agent to a site that is to be treated. The matrix can be coated with a coating or carrier comprising the RNAi agent, or the RNAi agent can be incorporated directly into the matrix compound, in which case the matrix acts as both the carrier and the scaffold.

Alternatively, a stent or stent graft can be used as a delivery vehicle. A stent is often used to support tissues while healing takes place. A vascular stent keeps blood vessels open after a surgical procedure. An intraluminal coronary artery stent is a small, self-expanding, stainless steel mesh tube that is placed within a coronary artery to keep the vessel open. It may be used during a coronary artery bypass graft surgery to keep the grafted vessel open, after balloon angioplasty to prevent reclosure of the blood vessel, or during other heart surgeries. Stents which are covered with a synthetic material such as dacron, PTFE or polyurethane are referred to as stent grafts. First developed to be used in peripheral arterial occlusive disease to prevent intimal hyperplasia or to cover small aneurysms or arteriovenous fistulae, the use of stent grafts has recently been expanded to treat aortic aneurysms with transcatheter techniques. There are two basic types of stent grafts, 1) stented grafts and 2) grafted stents. In stented grafts, a metal stent is attached to each end of the synthetic graft material. In grafted stents, the metal stent is covered by a synthetic graft material in its entire length.

RNAi Agents

Any RNAi agent that is capable of retarding or arresting the formation of restenosis, including blood clotting and/or intimal hyperplasia is appropriate for incorporation into the coating or carrier, and ultimately the therapeutic device of the present invention. Restenosis, including blood clotting and intimal hyperplasia, is caused by a cascade of events in response to vascular damage. As part of the inflammatory and reparative response to vascular damage, such as that resulting from vascular surgeries, inflammatory cells (e.g., monocytes, macrophages, and activated polymorphonuclear leukocytes and lymphocytes) often form inflammatory lesions in the blood vessel wall. Lesion formation activates cells in the intimal and medial cellular layers of the blood vessel or heart. The cellular activation may include the migration of cells to the innermost cellular layers, known as the intima. Such migrations pose a problem for the long-term success of vascular grafts because endothelial cells release smooth muscle cell growth factors (e.g., platelet-derived growth factor, interleukin-1, tumor necrosis factor, transforming growth factor-beta, and basic fibroblast growth factor), that cause these newly-migrated smooth muscle cells to proliferate. Additionally, thrombin has been demonstrated to promote smooth muscle cell proliferation both by acting as a growth factor itself and by enhancing the release of several other growth factors produced by platelets and endothelial cells (Wu et al., Annu. Rev. Med. 47:315-31 (1996)). Smooth muscle cell proliferation causes irregular and uncontrolled growth of the intima into the lumen of the blood vessel or heart, which constricts and often closes the vascular passage. Often, irregular calcium deposits in the media or lipid deposits in the intima accompany smooth muscle cell growths, such lipid deposits normally existing in the form of cholesterol and cholesteryl esters that are accumulated within macrophages, T lymphocytes, and smooth muscle cells. These calcium and lipid deposits cause arteriosclerotic hardening of the arteries and veins and eventual vascular failure. These arteriosclerotic lesions caused by vascular grafting can also be removed by additional reconstructive vascular surgery, but the failure rate of this approach due to restenosis has been observed to be between thirty and fifty percent.

Any RNAi agent that can interrupt or retard one or more of the elements of the above-described hyperplastic cascade is useful in practicing the present invention. Examples of useful RNAi agents include, but are not limited to, RNAi agents targeting genes involved in microtubule proliferation, extracellular matrix proliferation, platelet formation, the coagulation cascade, calcium channel genes, and genes that code for converting enzymes, cytokines, growth factors, smooth muscle proliferators, and the like. Representative genes are listed in Table 1.

TABLE 1

| | | | Entrez | |
|---|---|---|---|---|
| Gene | Synonyms | Full Name | Gene ID | Reference |
| TNFSF5 | IGM; IMD3; TRAP; | tumor necrosis | 959 | Cipollone at al., |
| | gp39; CD154; | factor (ligand) | | Circulation 108(22): |
| | CD40L; HIGM T- | superfamily, | | 2776-82 (2003). |
| | BAM; CD40LG; | member 5 | | |
| | hcd40L | | | |
| ACE | DCP; ACE1; | angiotensin I | 1636 | Taniguchi, et al., Jpn |
| | DCP1; CD143; | converting | | Circ J. 65(10):897- |
| | MGC26566 | enzyme | | 900(2001). |
| | | (peptidyl- | | |
| | | dipeptidase A) | | |
| | | 1 | | |
| ROCK1 | — | Rho-kinase bet | 397447 | Matsumoto, et al., |
| | | | | Arterioscler Thromb |
| | | | | Vasc Biol. 24(1): |
| | | | | 181-6 (2004) |
| ROCK2 | — | Rho-kinase bet | 397445 | Matsumoto, et al., |
| | | | | Arterioscler Thromb |
| | | | | Vasc Biol. 24(1): |
| | | | | 181-6 (2004) |
| Spp1 | OP; Eta; Opn; Ric; | Secreted | 20750 | Isoda, et al., Circ. |
| | BNSP; BSPI; | phosphoprotein | | Res. 91(1); 77-82 |
| | Opnl, Apl-i, ETA- | 1 | | (2002) |
| | 1; Spp-1; | | | |
| | minopontin, | | | |
| | osteopontin | | | |
| TP53 | P53; TRP53 | tumor protein | 7157 | Zee, et al., Hum |
| | | p53 | | Genet. 114(4): 386- |
| | | | | 90 (2004). |
| CCL2 | HC11; MCAF; | chemokine (C- | 6347 | Hokimoto, et al., |
| | MCP1; MCP-1; | C motif) ligand | | Circ. J. 66(1): 114-6 |
| | SCYA2; GDCF-2; | 2 | | (2002) |
| | SMC-CF; | | | |
| | MGC9434; GDCF- | | | |
| | 2 HC11 | | | |
| IL1B | IL-1; IL1F2; IL1- | interleukin 1, | 3553 | Marculescu, et al., |
| | BETA; | beta | | Thromb Haemost. |
| | NM_000575 | | | 90(3): 491-500 |
| | | | | (2003) |
| IL1RN | IRAP; IL1F3; | IL1RN | 3557 | Marculescu, et al., |
| | IL1RA; ICIL-1RA; | interleukin | | Thromb Haemost. |
| | MGC10430 | receptor | | 90(3): 491-500 |
| | | antagonist | | (2003) |
| ESR1 | ER; ESR; Era; | estrogen | 2099 | Ferrero, et al., |
| | ESRA; NR3A1 | receptor 1 | | Arterioscler Thromb |
| | | | | Vasc Biol. 23(12): |
| | | | | 2223-8 (2003) |
| Cyr61 | RGD:620763 | cysteine rich | 83476 | Grzeszkiewicz, et |
| | | protein 61 | | al., Endocrinology |
| | | | | 143(4): 1441-50 |
| | | | | (2002) |
| MMP3 | SL-1; STMY; | matrix | 4314 | Humphries, et al. |
| | STR1; STMY1; | metalloproteinase | | Euro Heart J. 23(9): |
| | TRANSIN | 3 | | 721-5 (2002) |
| ITGAV | CD51; MSK8; | integrin, alpha | 3685 | Sajid and Stouffer, |
| | VNRA | V | | Thromb Haemost. |
| | | | | 87(2): 187-93 (2002) |
| ITGB3 | CD61; GB3A | integrin, beta 3 | 3690 | Sajid and Stouffer, |
| | GPIIIa | | | Thromb Haemost. |
| | | | | 87(2): 187-93 (2002) |
| ITGA2B | GTA; CD41; | integrin, alpha | 3674 | Schafer, Tex Heart |
| | GP2B; CD41B; | 2b | | Inst. J. 24(2): 90-96 |
| | GBIIb | | | (1997). |
| Mmp2 | GelA; Clg4a; | matrix | 17390 | Kuzuya, et al., |
| | MMP-2 | metalloproteinase | | Circulation 108(11): |
| | | 2 | | 1375-81 (2003). |
| TGFB1 | CED; DPD1; | transforming | 7040 | Heine, et al., Kidney |
| | TGFB | growth factor, | | Int. 64(3): 1101-7 |
| | | beta 1 | | (2003). |
| PDGFA | PDGF1; PDFG-A | platelet-derived | 5154 | Carlin, et al., Am J |
| | | growth factor | | Physiol Lung Cell |
| | | alpha | | Mol Physiol. 284(6): |
| | | polypeptide | | L1020-6 (2003). |
| PDGFB | SIS; SSV; PDGF2; | platelet-derived | 5155 | Coombes, et al, J. |
| | c-sis | growth factor | | Infect Dis. 185(11): |
| | | beta | | 1621-30 (2002) |
| | | polypeptide | | |
| PDGFC | SCDGF | platelet-derived | 56034 | Fang, et al., |
| | | growth factor C | | Arterioscler Thromb |
| | | | | Vasc Biol. 24(4): |
| | | | | 787-92 (2004). |
| PDGFD | UEGFL NSTO036; | platelet-derived | 80310 | LaRochelle, et al., |
| | SCDGF-B; | factor D | | Cancer Res. |
| | MGC26867 | | | 62(9):2468-73 |
| | | | | (2002) |
| TGFA | — | transforming | 7039 | Maas-Szabowski, et |
| | | growth factor, | | al., J. Cell Sci. |
| | | alpha | | 116:2937-48 (2003) |
| FGA | — | fibrinogen, A | 2243 | Everse, Vox Sang. |
| | | alpha | | 83 Suppl 1:375-82 |
| | | polypeptide | | (2002). |
| FGB | — | firbrinogen, B | 2244 | Everse, Vox Sang. |
| | | polypeptide | | 83 Suppl 1:375-82 |
| | | | | (2002). |
| FGG | — | fibrinogen, | 2266 | Mosesson, J |
| | | gamma | | Thromb Haemost. |
| | | polypeptide | | 1(2): 231-8 (2003). |
| F2 | PT | coagulation | 2147 | De Cristofaro and |
| | | factor II | | De Candia, J |
| | | (thrombin) | | Thromb |
| | | | | Thrombolysis. 15(3): |
| | | | | 151-63 (2003) |
| IGF1 | — | isulin-like | 3479 | Duan, Mol Cell |
| | | growth factor | | Endocrinol. 206(1- |
| | | | | 2): 75-83 (2003) |
| MAPK3 | ERK1; PRKM3; | mitogen- | 5595 | Duan, Mol Cell |
| | P44ERK1; | activated | | Endocrinol. 206(1- |
| | P44MAPK | protein kinase | | 2): 75-83 (2003) |
| | | 3 | | |
| MAPK1 | ERK; p38; p40; | mitogen- | 5594 | Duan, Mol Cell |
| | p41; ERK2; ERT1; | activated | | Endocrinol. 206(1- |
| | MAPK2; PRKM1; | protein kinase | | 2): 75-83 (2003) |
| | PRKM2; | 1 | | |
| | P42MAPK; | | | |
| | p41 mapk | | | |
| EGF | — | epidermal | 1950 | Pastore, et al., |
| | | growth factor | | Circulation |
| | | | | Research. 77:51 9- |
| | | | | 529 (1995). |
| EFGR | ERBB; Mena; | epidermal | 1956 | Pastore, et al., |
| | ERBB1 | growth factor | | Circulation |
| | | receptor | | Research. 77:519- |
| | | | | 529 (1995). |
| FGF1 | AFGF; ECGF; | fibroblast | 2246 | Hughes, et al. |
| | FGFA; ECGFA; | growth factor | | Cardiovasc Res. |
| | ECGFB; HBGF1; | (acidic) | | 27:1214-1219 |
| | GLIO703; ECGF- | | | (1993). |
| | beta; EGE-apha | | | |
| FGF2 | BFGF; FGFB; | fibroblast | 2247 | Casscells, et al., |
| | HBGH-2 | growth factor | | Proc Natl Acad Sci |
| | | (basic) | | USA. 89(15): 7159- |
| | | | | 7163 (1992). |
| FGFR1 | H2; H3; H4; H5; | fibroblast | 2260 | Li, et al. Circulation |
| | CEK; FLG; FLT2; | growth factor | | 106:854 (2002) |
| | KAL2; BFGFR; C- | receptor 1 | | |
| | FGR; N-SAM | | | |

Platelet derived growth factor (PGDF) is composed of two distinct polypeptide chains, A and B, that form homodimers (AA or BB) or heterodimers (AB). PGDF receptors have intrinsic tyrosine kinase activity. Following auto phosphorylation of the PGDF receptor, numerous signal-transducing proteins associate with the receptor and are subsequently tyrosine phosphorylated. Proliferative responses to PDGF action are exerted on many mesenchymal cell types.

Epidermal growth factor (EGF) binds to specific high-affinity, low-capacity receptors on the surface of responsive cells. Like PGDF, intrinsic to the EGF receptor is tyrosine kinase activity, which is activated in response to EGF binding. The kinase domain of the EGF receptor phosphorylates the EGF receptor itself (autophosphorylation) as well as other proteins in signal transduction cascades that associate with the receptor following activation. EGF has proliferative effects on cells of both mesodermal and ectodermal origin, particularly keratinocytes and fibroblasts.

There are at least 19 distinct members of the fibroblast growth factor (FGF) family. The two originally characterized FGFs were designated FGF1 (aka acidic FGF or aFGF) and FGF2 (basic-FGF or bFGF). FGFs interact with specific cell-surface receptors. Four distinct receptor types have been identified: FGFR-1, FGFR-2, FGFR3 and FGFR-4. Each of these receptors has intrinsic tyrosine kinase activity like the PDGF and EGF receptors. As with all transmembrance receptors that have tyrosine kinase activity, autophosphorylation of the receptor is the immediate response to FGF binding. FGF receptors are widely expressed in developing bone.

Transforming growth factor beta (TGF-β) was originally characterized as a protein capable of inducing a transformed phenotype in non-neoplastic cells in culture. This effect was reversible, as demonstrated by the reversion of the cells to a normal phenotype following removal of the TGF-β. Subsequently, many proteins homologous to TGF-β have been identified, sharing extensive regions of similarity in amino acid sequence. The TFG-β related family of proteins includes the activin and inhibin proteins, and may comprise as many as 100 different proteins. There are several classes of cell-surface receptors that bind different TGF-βs with differing affinities. There also are cell-type specific differences in receptor sub-types. Unlike the EGF, PDGF and FGF receptors, the TGF-β family of receptors all have intrinsic serine/threonine kinase activity and, therefore, induce distinct cascades of signal transduction. TGF-βs have proliferative effects on many mesenchymal and epithelial cell types.

Transforming growth factor alpha (TGF-α), like the beta form, was first identified as a substance secreted from certain tumor cells that, in conjunction with TGF-β-1, could reversibly transform certain types of normal cells in culture. TGF-α binds to the EGF receptor, as well as its own distinct receptor, and it is this interaction that is thought to be responsible for the growth factor's effect. The predominant sources of TGF-α are carcinomas, but activated macrophages and keratinocytes (and other epithelial cells) are secrete TGF-α. In normal cells, TGF-α is a potent keratinocyte growth factor.

Insulin-like growth factor I (IGF-I) is a growth factor structurally related to insulin. IGF-I is the primary protein involved in responses of cells to growth hormone; that is, IGF-I is produced in response to growth hormone and then induces subsequent cellular activities. Subsequent studies have demonstrated that IGF-I has autocrine and paracrine activities in addition to the initially observed endocrine activities on bone.

Fibrin is the protein responsible for the clotting of blood. It is a fibrillar protein that spontaneously polymerizes to form a mesh that covers a wound. Fibrin is made from fibrinogen, a soluble plasma protein that is produced by the liver. Processes in the coagulation cascade activate the enzyme thrombin, which is responsible for converting fibrinogen into fibrin. Fibrin can then polymerise and form a clot. Processes in the coagulation cascade activate a protease enzyme known as thrombin (or activated Factor 11). Thrombin cleaves the fibrinopeptides off the alpha and beta chains of fibrinogen, converting it to fibrin.

Thrombin is not a normal constituant of the circulating blood, but is generated by the catalytic cleavage of its plasma precursor, prothrombin (factor II), by the activated Stuart factor (factor Xa). This is the final step of one or both of the two convergent chains of reactions called the intrinsic and extrinsic pathways of coagulation. The transformation requires the presence of an activated cofactor, factor Va, released from factor V by thrombin itself, and whose binding to prothrombin accelerates the activity of factor Xa in a non-enzymatic manner. Thrombin is a glycoprotein formed by two peptides chains of 36 and 259 amino-acids linked by disulfide bonds. The earliest identified function of thrombin is the cleavage of fibrinogen into fibrin monomers and the activation of the fibrin-stabilizing factor (factor XIII) and protein C. Thrombin has the property of activating factor XIII to act as a transaminase and form covalent links between the carboxyl and amino groups of two different fibrin monomers, enhancing the strength of the clot. Thrombin is, however, more than a simple plasma enzyme. Its properties to stimulate platelets and cause them to expand, aggregate and release components of the alpha and dense granules have been recognized for years. Thrombin also has numerous effects on various cells, some of them being of major importance in the development of LPS-induced liver injury. This results in increased production of the platelet-derived growth factor (PDGF), factor XIII, factor VIII, tPA, PAI, platelet activating factor (PAF), modification of the interactions between endothelial cells and the underlying matrix or between endothelial cells, and the expression of adhesion glycoproteins to the cell surface, thereby increasing the binding of inflamatory cells to the endothelium. Thrombin induces chemotaxis in neutrophils and promotes the release of inflammatory components. Thrombin is also a potent chemotaxin for macrophages, and can alter their production of cytokines and arachidonic acid metabolites.

Platelet activating factor is a lipid-acetyl glycerol ether phosphocholine derived from a cell membrane constituent, glycerophorylcholine. Platelet activating factor can be produced from a range of inflammatory cells and has an enormous range of functions which mirror almost every facet of inflammation. Glucocorticoids may exert their anti-inflammatory role by their activation of lipocortin, a peptide inhibitor of phospholipase A2. Phospholipase A2 is involved in the early stages of platelet activating factor production. Platelet activating factor is a potent platelet aggregating agent and inducer of systemic anaphylactic symptoms, including hypotension, thrombocytopenia, neutropenia, and bronchoconstriction.

Other agents that are useful in conjunction with the present invention will be readily apparent to those of skill in the art.

It should also be understood that the present invention contemplates targeting one or more than one target sequence, including one or more than one sequence set forth in Table 1. Furthermore, the present invention contemplates targeting a restenosis-associated sequence as described herein in combination with one or more sequences associated with other aspects of cardiovascular disease including sequences involved in cholesterol metabolism, blood pressure homeostasis, lipid metabolism and the like.

Incorporation of RNAi Agents into Carriers, Coatings or Matrices

As stated previously, the RNAi agents, whether siRNAs or ddRNAs, are formulated with a carrier or coating, which may then be associated with a delivery vehicle such as a matrix or stent graft, ultimately forming a therapeutic device. The therapeutic device may be the carrier itself that is a flowable liquid or gel that is applied to the exterior of a blood vessel (see, e.g., U.S. Pat. No. 6,730,313). Alternatively, the therapeutic device may be a synthetic or naturally-occurring matrix that is formulated with the RNAi agent directly, or the therapeutic device may be a synthetic or naturally-occurring matrix that is coated with the RNAi agent/carrier or coating. Either matrix would be applied to the exterior of a blood vessel. In yet another embodiment, the therapeutic device may be a vascular prosthesis that is implanted in a blood vessel such as a stent or stent graft. The graft portion of the stent may be formulated to carry or comprise the RBAi agent directly; alternatively, the stent or stent graft may be coated with the carrier comprising the ddRNAi agent.

The RNAi agents useful in practicing the present invention can be incorporated into carriers or coatings which are then immobilized or adhered to a delivery vehicle to form a therapeutic device. Methods for incorporating RNAi agents into a carrier or coating include, but are not limited to, covalent attachment of the RNAi agent with the coating or non-covalent interaction of the RNAi agent with the carrier by using an electrostatic or an ionic attraction between a charged RNAi agent and a component of the coating bearing a complementary charge. The RNAi agents also can be admixed, and not otherwise interact with the carrier or coating. The carriers or coatings also can be fabricated to incorporate the RNAi agents into reservoirs located in the coating. The reservoirs can have a variety of shapes and sizes and they can be produced by an array of methods. For example, the reservoir can be a monolithic structure located in one or more components of the coating. Alternatively, the reservoir can be made up of numerous small microcapsules that are, for example, embedded in the material from which the coating is fabricated. Furthermore, the reservoir can be a coating that includes the RNAi agent diffused throughout, or within a portion of the coating's three-dimensional structure. The reservoirs can be porous structures that allow the RNAi agent to be slowly released from its encapsulation, or the reservoir can include a material that bioerodes following implantation and allows the drug to be released in a controlled fashion.

Stents that have been formulated to delivery therapeutic agents as known in the art. For example, U.S. Pat. No. 5,163,952 to Froix discloses a thermal memoried expanding plastic stent device, which can be formulated to carry a medicinal agent by utilizing the material of the stent itself as an inert polymeric drug carrier. Pinchuk, in U.S. Pat. No. 5,092,877, discloses a stent of a polymeric material which can be employed with a coating that provides for the delivery of drugs. Ding, et al., U.S. Pat. No. 5,837,313 disclose a method of coating an implantable open lattice metallic stent prosthesis with a drug releasing coating. Patents directed to devices of the class utilizing biodegradable or biosorbable polymers include, for example, Tang, et al., U.S. Pat. No. 4,916,193, and MacGregor, U.S. Pat. No. 4,994,071. Sahatjian in U.S. Pat. No. 5,304,121, discloses a coating applied to a stent consisting of a hydrogel polymer and a preselected drug; possible drugs include cell growth inhibitors and heparin. Drugs have also been delivered to the interior of vascular structures by means of a polyurethane coating on a stent, where the coating was swelled and a biologically active compound was incorporated within the interstices of the polymer (Lambert, U.S. Pat. No. 5,900,246).

In another embodiment of a delivery devide, an RNAi agent may be delivered to an extraluminal site adjacent to the point of vascular injury by means of an implanted infusion pump or biodegradable vehicle (for example, see Edelman, et al., U.S. Pat. No. 5,527,532). In one embodiment of the Edelman invention, the biodegradable vehicle is implanted in the adventitia at a site adjacent to the site of injury, where a therapeutic is delivered to the adventitia and from the aventitia to exterior surface of the vascular wall.

Reversibly Associated RNAi Agents

In one embodiment of the present invention, if it is desired that the RNAi agent is first associated with a carrier or coating in order to prepare the therapeutic device, but then the RNAi agent or agents is released from the carrier or coating in a controlled manner once the therapeutic device has been implanted or associated with a blood vessel (a reversibly-associated RNAi agent). Such a reversibly associated RNAi agent can, for example, be entrapped in a carrier, coating or matrix by adding the agent to the carrier, coating or matrix components during manufacture of the carrier, coating or matrix. In an exemplary embodiment, the RNAi agent is added to a polymer melt or a solution of the polymer. Other methods for reversibly incorporating RNAi agents into a delivery matrix will be apparent to those of skill in the art.

Examples of such reversible associations include, for example, RNAi agents that are mechanically entrapped within the carrier, coating or matrix and RNAi agents that are encapsulated in structures (e.g., within microspheres, liposomes, etc.) that are themselves entrapped in, or immobilized on, the carrier, coating or matrix. Other reversible associations include, but are not limited to, RNAi agents that are adventitiously adhered to the carrier, coating or matrix by, for example, hydrophobic or ionic interactions and RNAi agents bound to one or more carrier, coating or matrix component by means of a linker cleaved by one or more biologically relevant processes. The reversibly-associated RNAi agents can be exposed on the coating surface or they can be covered with the same or a different carrier or coating, such as a bioerodable polymer, as described below.

In one embodiment, the surface character of the carrier, coating or matrix material is altered or manipulated by including certain additives or modifiers in the coating material during its manufacture. A method of preparing surface-functionalized polymeric materials by this method is set forth in, for example, U.S. Pat. No. 5,784,164 to Caldwell. In the Caldwell method, additives or modifiers are combined with the polymeric material during its manufacture. These additives or modifiers include compounds that have affinity sites, compounds that facilitate the controlled release of agents from the polymeric material into the surrounding environment, catalysts, compounds that promote adhesion between the bioactive materials and the coating material and compounds that alter the surface chemistry of the coating material.

As used herein, the term “affinity site” refers to a site on the polymer that interacts with a complementary site on the RNAi agent, or on the exterior surface of the delivery vehicle to which the carrier, coating or matrix is applied. Affinity sites for the RNAi agent, carrier, or delivery vehicles that are contemplated in the practice of the present invention include such functional groups as hydroxyl, carboxyl, carboxylic acid, amine groups, hydrophobic groups, inclusion moieties (e.g., cyclodextrin, complexing agents), biomolecules (e.g. antibodies, haptens, saccharides, peptides) and the like, that promote physical and/or chemical interaction with the RNAi agent. In such an embodiment, the affinity site interacts with the RNAi agent by non-covalent means. The particular compound employed as the modifier will depend on the chemical functionality of the RNAi agent and the groups on the carrier, coating or matrix. Appropriate functional groups for a particular purpose can be deduced by one of skill in the art.

In another embodiment, the coating used in the invention is a substantially flowable material that can be delivered to the vascular site by means of, for example, a catheter, needle or other percutaneous delivery device. Preferred embodiments of the substantially flowable material are those that cure to a substantially non-flowable coating in vivo. In this case, the carrier itself is the delivery vehicle, and the RNAi agent/carrier combination is the therapeutic device. Materials meeting the flowably/curable criteria include, for example, fibrin sealants, hydrophobic poly(hydroxy acids) and the like. The amount of the RNAi agent contained in the substantially flowable material varies depending on a number of factors, including, for example, the activity of the particular RNAi agent or agents being delivered and the tenaciousness with which the RNAi agent adheres to the carrier, coating or matrix.

In another embodiment, the RNAi agent interacts with a surfactant that adheres to the carrier, coating or matrix material. Presently preferred surfactants are selected from benzalkonium halides and sterylalkonium halides. Other surfactants suitable for use in the present invention are known to those of skill in the art.

Covalently Attached RNAi Agents

In another embodiment, the RNAi agent is covalently bonded to a reactive group located on one or more components of the carrier or coating. The art is replete with methods for preparing derivatized, polymerizable monomers, attaching nucleic acids onto polymeric surfaces and derivatizing nucleic acids and polymers to allow for this attachment (see, for example, Hermanson, Bioconjugate Techniques, Academic Press, 1996, and references therein). Common approaches include the use of coupling agents such as glutaraldehyde, cyanogen bromide, p-benzoquinone, succinic anhydrides, carbodiimides, diisocyanates, ethyl chloroformate, dipyridyl disulfide, epichlorohydrin, azides, among others, which serve as attachment vehicles for coupling reactive groups of derivatized nucleic acid molecules to reactive groups on a monomer or a polymer.

A polymer can be functionalized with reactive groups by, for example, including a moiety bearing a reactive group as an additive to a blend during manufacture of the polymer or polymer precursor. The additive is dispersed throughout the polymer matrix, but does not form an integral part of the polymeric backbone. In this embodiment, the surface of the polymeric material is altered or manipulated by the choice of additive or modifier characteristics. The reactive groups of the additive are used to bind the one or more RNAi agents to the polymer.

A useful method for preparing surface-functionalized polymeric materials by this method is set forth in, for example, Caldwell, supra. In the Caldwell method, additives or modifiers are combined with the polymeric material during its manufacture. These additives or modifiers include compounds that have reactive sites, compounds that facilitate the controlled release of agents from the polymeric material into the surrounding environment, catalysts, compounds that promote adhesion between bioactive materials (such as an RNAi agent) and the polymeric material and compounds that alter the surface chemistry of the polymeric material. In another embodiment, polymerizable monomers bearing reactive groups are incorporated in the polymerization mixture. The functionalized monomers form part of the polymeric backbone and, preferably, present their reactive groups on the surface of the polymer.

Reactive groups contemplated in the practice of the present invention include functional groups, such as hydroxyl, carboxyl, carboxylic acid, amine groups, and the like, that promote physical and/or chemical interaction with the bioactive material. The particular compound employed as the modifier will depend on the chemical functionality of the biologically active agent and can readily be deduced by one of skill in the art. In the present embodiment, the reactive site binds a bioactive agent by covalent means. It will, however, be apparent to those of skill in the art that these reactive groups can also be used to adhere the RNAi agents to the polymer by hydrophobic/hydrophilic, ionic and other non-covalent mechanisms.

In addition to manipulating the composition and structure of the polymer during manufacture, a preferred polymer can also be modified using a surface derivitization technique. There are a number of surface-derivatization techniques appropriate for use in fabricating the RNAi agent/carrier and, ultimately, the therapeutic devices of the present invention. These techniques for creating functionalized polymeric surfaces (e.g., grafting techniques) are well known to those skilled in the art. For example, techniques based on eerie ion initiation, ozone exposure, corona discharge, UV irradiation and ionizing radiation (⁶⁰Co, X-rays, high energy electrons, plasma gas discharge) are known and can be used in the practice of the present invention.

Substantially any reactive group that can be reacted with a complementary component on an RNAi agent can be incorporated into a polymer and used to covalently attach the RNAi agent to the carrier coating of use in the invention. In a preferred embodiment, the reactive group is selected from amine-containing groups, hydroxyl groups, carboxyl groups, carbonyl groups, and combinations thereof. In a further preferred embodiment, the reactive group is an amino group.

Aminated polymeric materials useful in practicing the present invention can be readily produced through a number of methods well known in the art. For example, amines may be introduced into a preformed polymer by plasma treatment of materials with ammonia gas as found in Holmes and Schwartz, Composites Science and Technology, 38: 1-21 (1990). Alternatively, amines can be provided by grafting acrylamide to the polymer followed by chemical modification to introduce amine moieties by methods well known to those skilled in the art; e.g., by the Hofmann rearrangement reaction. Also, grafted acrylamide-containing polymer may be prepared by radiation grafting as set forth in U.S. Pat. No. 3,826,678 to Hoffman, et al. A grafted N-(3-aminopropyl)methacrylamide-containing polymer may be prepared by ceric ion grafting as set forth in U.S. Pat. No. 5,344,455 to Keogh et al. Polyvinylamines or polyalkylimines also can be covalently attached to polyurethane surfaces according to the method taught by U.S. Pat. No. 4,521,564 to Solomone, et al. Alternatively, for example, aminosilane may be attached to the surface as set forth in U.S. Pat. No. 5,053,048 to Pinchuk.

In yet another embodiment, a polymeric coating material, or a precursor material is exposed to a high frequency plasma with microwaves or, alternatively, to a high frequency plasma combined with magnetic field support to yield the desired reactive surfaces bearing at least a substantial portion of reactant amino groups upon the substrate to be derivatized with the RNAi agent.

A functionalized carrier or coating surface also can be prepared by, for example, first submitting a carrier coating component to a chemical oxidation step. This chemical oxidation step is then followed, for example, by exposing the oxidized substrate to one or more plasma gases containing ammonia and/or organic amine(s) which react with the treated surface. In one embodiment, the gas is selected from the group consisting of ammonia, organic amines, nitrous oxide, nitrogen, and combinations thereof. The nitrogen-containing moieties derived from this gas are preferably selected from amino groups, amido groups, urethane groups, urea groups, and combinations thereof, more preferably primary amino groups, secondary amino groups, and combinations thereof. In another aspect of this embodiment, the nitrogen source is an organic amine. Examples of suitable organic amines include, but are not limited to, methylamine, dimethylamine, ethylamine, diethylamine, ethylmethylamine, n-propylamine, allylamine, isopropylamine, n-butylamine, n-butylmethylamine, n-amylamine, n-hexylamine, 2-ethylhexylamine, ethylenediamine, 1,4-butanediamine, 1,6-hexanediamine, cyclohexylamine, n-methylcyclohexylamine, ethyleneimine, and the like. In a further aspect, the chemical oxidation step is supplemented with, or replaced by, submitting the polymer to one or more exposures to plasma-gas that contains oxygen. In yet a further preferred embodiment, the oxygen-containing plasma gas further contains argon (Ar) gas to generate free radicals. Immediately after a first-step plasma treatment with oxygen-containing gases, or oxygen/argon plasma gas combinations, the oxidized polymer is preferably functionalized with amine groups. As mentioned above, functionalization with amines can be performed with plasma gases such as ammonia, volatile organic amines, or mixtures thereof.

In an exemplary embodiment utilizing ammonia and/or organic amines, or mixtures thereof, as the plasma gases, a frequency in the radio frequency (RF) range of from about 13.0 MHz to about 14.0 MHz is used. A generating power of from 0.1 Watts per square centimeter to about 0.5 Watts per square centimeter of surface area of the electrodes of the plasma apparatus is preferably utilized. An exemplary plasma treatment includes evacuating the plasma reaction chamber to a desired base pressure of from about 10 to about 50 mTorr. After the chamber is stabilized to a desired working pressure, ammonia and/or organic amine gases are introduced into the chamber. Preferred flow rates are typically from about 200 to about 650 standard mL per minute. Typical gas pressure ranges from about 0.01 to about 0.5 Torr, and preferably from about 0.2 to about 0.4 Torr. A current having the desired frequency and level of power is supplied by means of electrodes from a suitable external power source. Power output is up to about 500 Watts, preferably from about 100 to about 400 Watts. The plasma treatment can be performed by means of a continuous or batch process.

Optimization procedures for the plasma treatment and the effect of these procedures on the characteristics and the performance of the reactive polymers can be determined by, for example, evaluating the extent of substrate functionalization. Methods for characterizing functionalized polymers are well known in the art.

The result of the above-described exemplary methods is preferably a polymeric surface that contains a significant number of primary and/or secondary amino groups. These groups are preferably readily reactive at room temperature with an inherent, or an appended, reactive functional group on the RNAi agents. Once the amine-containing polymeric carrier coating is prepared, it can be used to covalently bind the RNAi agents using a variety of functional groups including, for example, ketones, aldehydes, activated carboxyl groups (e.g. activated esters), alkyl halides and the like.

Synthesis of specific RNAi agent/carrier conjugates is generally accomplished by: 1) providing a carrier or coating component comprising an activated polymer, such as an acrylic acid, and an RNAi agent having a position thereon which will allow a linkage to form; 2) reacting the complementary substituents of the RNAi agent and the carrier coating componenf in an inert solvent, such as methylene chloride, chloroform or DMF, in the presence of a coupling reagent, such as 1,3-diisopropylcarbodiimide or any suitable dialkyl carbodiimide (Sigma Chemical), and a base, such as dimethylaminopyridine, diisopropyl ethylamine, pyridine, triethylamine, etc. Alternative specific syntheses are readily accessible to those of skill in the art (see, for example, Greenwald, et al., U.S. Pat. No. 5,880,131).

One skilled in the art understands that in the synthesis of compounds useful in practicing the present invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to Protective Groups in Organic Chemistry, McOmie, ed., Plenum Press, NY, N.Y. (1973); and, Protective Groups in Organic Synthesis, Greene, ed., John Wiley & Sons, NY (1981) for the teaching of protective groups which may be useful in the preparation of compounds of the present invention.

Delivery Vehicle Formats

The present invention includes providing a therapeutic device to treat restenosis. In one embodiment, the site of insult is covered or partially covered with a flowable liquid or semi-solid liquid comprising an RNAi agent, where the RNAi agent/carrier combination itself is the therapeutic device. In another embodiment, one or more RNAi agents are associated with a carrier or coating, which is then associated with a delivery vehicle such as a matrix, stent, or stent graft to form a therapeutic device. The carrier or coating can take a number of forms. For example, as described herein, useful carriers or coatings can be in the form of foams, gels, suspensions, microcapsules, solid polymeric materials and fibrous or porous structures. Stents generally are made of metal alloys or other deformably rigid materials, and grafts are generally made of polymeric material. The carrier, coating or matrix can be multilayered with one or more of the layers including an RNAi agent. Moreover, a carrier or coating can be layered on a component impregnated with the RNAi agent. Many materials that are appropriate for use as carriers or coatings or matrices or grafts in the present methods are known in the art and both natural and synthetic materials are useful in practicing the present invention.

Selection of Carrier, Coating, Matrix or Graft Materials

Suitable polymers that can be used as carrier, coating, matrix or graft material in the present invention include, but are not limited to, water-soluble and water-insoluble, biodegradable, bioerodable or nonbiodegradable polymers. The carrier, coating, matrix or graft is preferably sufficiently porous, or capable of becoming sufficiently porous, to permit efflux of the RNAi agents from the coating. The carrier, coating, matrix or graft also preferably is sufficiently non-inflammatory and is biocompatible so that inflammatory responses do not prevent the delivery of the RNAi agents to the vascular tissue. It is advantageous if the carrier, coating, matrix or graft also provides at least partial protection of the RNAi agents from the adverse effects of nucleases and other relevant degradative species. In addition, it is advantageous for the carrier, coating, matrix or graft to produce controlled, sustained delivery of the one or more RNAi agents.

Many polymers can be utilized to form the carrier, coating, matrix or graft. A carrier, coating, or matrix can be, for example, a gel, such as a hydrogel, organogel or thermoreversible gel. Other useful polymer types include, but are not limited to, thermoplastics and films. Moreover, the carrier, coating, or matrix can comprise a homopolymer, copolymer or a blend of these polymer types. The carrier, coating, or matrix can also include an RNAi agent-loaded microparticle dispersed within a component of the carrier, coating, or matrix, which serves as a dispersant for the microparticles. Microparticles include, for example, microspheres, microcapsules and liposomes.

The carrier, coating, or matrix can serve to immobilize the microparticles at a particular site, enhancing targeted delivery of the encapsulated RNAi agents. Rapidly bioerodible polymers such as polylactide-co-glycolide, polyanhydrides, and polyorthoesters, whose carboxylic groups are exposed on the surface are useful in the coatings of use in the invention. In addition, polymers containing labile bonds, such as polyesters, are well known for their hydrolytic reactivity. The hydrolytic degradation rates of the carrier, coating, or matrix can generally be altered by simple changes in the polymer backbone.

The carrier, coating, or matrix can be made up of natural and/or synthetic polymeric materials. Representative natural polymers of use as coatings in the present invention include, but are not limited to, proteins, such as zein, modified zein, casein, gelatin, gluten, serum albumin, or collagen, and polysaccharides, such as cellulose, dextrans, and polyhyaluronic acid. Also of use in practicing the present invention are materials, such as collagen and gelatin, which have been widely used on implantable devices, such as textile grafts (see, for example, Hoffman, et al., U.S. Pat. No. 4,842,575, and U.S. Pat. No. 5,034,265), but which have not been utilized as components of adherent coatings for periadventitial delivery of RNAi agents, such as those preventing or retarding the development of restenosis. Hydrogel or sol-gel mixtures of polysaccharides are also known. Furthermore, fibrin, an insoluble protein formed during the blood clotting process, has also been used as a sealant for porous implantable devices (see, for example, Sawhey, et al., U.S. Pat. No. 5,900,245). Useful fibrin sealant compositions are disclosed in, for example, Edwardson, et al., U.S. Pat. No. 5,770,194, and U.S. Pat. No. 5,739,288. These and other naturally based agents, alone or in combination, can be used as a carrier, coating, or matrix in practicing the present invention.

The carrier, coating, matrix or graft portion of a stent graft may comprise a synthetic polymer. Representative synthetic polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Also, the carrier, coating, matrix or graft portion of a stent graft may comprise a synthetically-modified natural polymer. Synthetically modified natural polymers include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Biodegradable and Bioresorbable Carrier, Coating, Matrix or Graft Materials

RNAi agents in combination with a carrier, coating, matrix or graft may have intrinsic and controllable biodegradability, if desired, so that the RNAi agents persist for about a week to about six months or longer. The carriers, coatings, matrices or grafts also are preferably biocompatible, non-toxic, contain no significantly toxic monomers and degrade into non-toxic components. Moreover, preferred carriers, coatings, matrices or grafts are chemically compatible with the RNAi agents to be delivered, and tend not to denature the RNAi agents. Still further preferred carriers, coatings, matrices or grafts are, or become, sufficiently porous to allow the incorporation of RNAi agents and their subsequent liberation from the coating by diffusion, erosion or a combination thereof. The carriers, coatings, matrices or grafts should also remain at the site of application by adherence or by geometric factors, such as by being formed in place or softened and subsequently molded or formed into fabrics, wraps, gauzes, particles (e.g., microparticles), and the like, or in the case of a graft, being associated with an implanted stent. Types of monomers, macromers, and polymers that can be used are described in more detail below.

Representative biodegradable polymers include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

Preferred carriers, coatings, or matrices (and, in some instances, grafts) are water-insoluble materials that comprise within at least a portion of their structure, a bioresorbable molecule. An example of such a carrier, coating, matrix or graft is one that includes a water-insoluble copolymer, which has a bioresorbable region, a hydrophilic region and a plurality of crosslinkable functional groups per polymer chain.

For purposes of the present invention, “water-insoluble materials” includes copolymers that are substantially insoluble in water or water-containing environments. Thus, although certain regions or segments of the copolymer may be hydrophilic or even water-soluble, the copolymer molecule, as a whole, does not by any substantial measure dissolve in water or water-containing environments.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region is preferably hydrophobic. In another embodiment, however, the bioresorbable region may be designed to be hydrophilic so long as the copolymer composition as a whole is not rendered water-soluble. Thus, the bioresorbable region is designed based on the preference that the copolymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable carriers, coatings, matrices or grafts include, for example, synthetically produced resorbable block copolymers of poly(α-hydroxy-carboxylic acid)/poly(oxyalkylene, (see, Cohn, et al., U.S. Pat. No. 4,826,945). These copolymers are not crosslinked and are water-soluble so that the body can excrete the degraded block copolymer compositions. See, Younes, et al., J. Biomed. Mater. Res. 21: 1301-1316 (1987); and Cohn, et al. J. Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly (amino acids), poly(anhydrides), poly(orthoesters), poly(carbonates), poly(phosphazines), poly(phosphoesters), poly(thioesters), polysaccharides and mixtures thereof. In some embodiments, the biosresorbable polymer includes a poly(hydroxy) acid component. Of the poly(hydroxy) acids, Polyiactic acid, polyglycolic acid, polycaproic acid, polybutyric acid, polyvaleric acid and copolymers and mixtures thereof are preferred. In addition to forming fragments that are absorbed in vivo (“bioresorbed”), some polymeric coatings for use in the methods of the invention can also form an excretable and/or metabolizable fragment.

Higher order copolymers can also be used as carriers, coatings, matrices or grafts in the methods of the present invention. For example, Casey et al, U.S. Pat. No. 4,438,253 discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other coatings based on lactic and/or glycolic acids can also be utilized. For example, Spinu, U.S. Pat. No. 5,202,413, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a difunctional compound, such as, a diisocyanate, diacylchloride or dichlorosilane.

The monomers, polymers and copolymers of use in the present invention may, in some embodiments, form a stable aqueous emulsion, and more preferably a flowable liquid. The relative proportions or ratios of the bioresorbable and hydrophilic regions, respectively, are preferably selected to render the block copolymer composition water-insoluble. Furthermore, these compositions are preferably sufficiently hydrophilic to form a hydrogel in aqueous environments when crosslinked.

The specific ratio of the two regions of the block copolymer composition for use as carriers, coatings, matrices or grafts in the present invention will vary depending upon the intended application and will be affected by the desired physical properties of the implantable coating, the site of implantation, as well as other factors. For example, the composition of the present invention will preferably remain substantially water-insoluble when the ratio of the water-insoluble region to the hydrophilic region is from about 10:1 to about 1:1, on a percent by weight basis.

Bioresorbable regions of carriers, coatings, matrices or grafts useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes. As set forth above, the some compositions also include a hydrophilic region. Although some compositions contain a hydrophilic region, in other coatings, this region is designed and/or selected so that the composition as a whole, remains substantiany water-insoluble.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene) oxide. Such poly(alkylene) oxides can include, for example, poly(ethylene) oxide, poly(propylene) oxide and mixtures and copolymers thereof.

Concerning the disposition of the RNAi agents in the carriers, coatings, matrices or grafts, substantially any combination of RNAi agent and carriers, coatings, matrices or grafts that is of use in achieving the object of the present invention is contemplated by this invention. In some embodiments, the RNAi agent is dispersed in a resorbable coating that imparts controlled release properties to the RNAi agent. The controlled release properties can result from, for example, a resorbable polymer that is cross-linked with a degradable cross-linking agent. Alternatively, the controlled release properties can arise from a resorbable polymer that incorporates the RNAi agent in a network of pores formed during the cross-linking process or gelling. In another embodiment, the RNAi agent is loaded into microspheres, which are themselves biodegradable and the microspheres are embedded in the carriers, coatings, matrices or grafts. Many other appropriate RNAi agentlcoating/matrixlgraft formats will be apparent to those of skill in the art.

In another preferred embodiment, an underlying polymeric component of a carrier, coating, matrix or graft of use in the invention is first impregnated with the RNAi agent and a resorbable polymer is the layered onto the underlying component. In this embodiment, the impregnated component serves as a reservoir for the RNAi agent, which can diffuse out through pores in a resorbable polymer network, through voids in a polymer network created as a resorbable polymer degrades in vivo, or through a layer of a gel-like coating. Other controlled release formats utilizing a polymeric substrate, an RNAi agent and a carrier, coating, matrix or graft will be apparent to those of skill in the art.

Hydrogel-Based Carriers, Coatings, Matrices or Grafts

Also contemplated for use in the practice of the present invention as a carrier or coating composition are hydrogels. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell, et al., U.S. Pat. No. 5,410,016, and U.S. Pat. No. 5,529,914, disclose water-soluble systems, which are crosslinked block copolymers having a water-soluble central block segment sandwiched between two hydrolytically labile extensions. Such copolymers are further end-capped with photopolymerizable acrylate functionalities. When crosslinked, these systems become hydrogels. The water soluble central block of such copolymers can include poly(ethylene glycol); whereas, the hydrolytically labile extensions can be a poly(.alpha.-hydroxy acid), such as polyglycolic acid or polylactic acid. See, Sawhney et al., Macromolecules 26: 581-587 (1993).

In yet another embodiment, the RNAi agent is dispersed in a hydrogel that is cross-linked to a degree sufficient to impart controlled release properties to the RNAi agent. The controlled release properties can result from, for example, a hydrogel that is cross-linked with a degradable cross-linking agent. Alternatively, the controlled release properties can arise from a hydrogel that incorporates the RNAi agent in a network of pores formed during the cross-linking process.

In another preferred embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another embodiment, a component of the carrier, coating, matrix or graft is first impregnated with the RNAi agent and a hydrogel is layered onto the impregnated coating component. In this embodiment, the impregnated coating component serves as a reservoir for the RNAi agent, which can diffuse out through pores in the hydrogel network or, alternatively, can diffuse out through voids in the network created as the hydrogel degrades in vivo (see, for example, Ding, et al., U.S. Pat. No. 5,879,697 and U.S. Pat. No. 5,837,313). Other controlled release formats utilizing a polymeric substrate, an RNAi agent and a hydrogel will be apparent to those of skill in the art.

As set forth above, useful carriers, coatings, matrices or grafts of the present invention can also include a plurality of crosslinkable functional groups. Any crosslinkable functional group can be incorporated into these compositions so long as it permits or facilitates the formation of a hydrogel. Preferably, the crosslinkable functional groups of the present invention are olefinically unsaturated groups. Suitable olefinically unsaturated functional groups include without limitation, for example, acrylates, methacrylates, butenates, maleates, allyl ethers, allyl thioesters and N-allyl carbamates. In some embodiments, the crosslinking agent is a free radical initiator, such as for example, 2,2′-azobis (N,N′dimethyleneisobutyramidine) dihydrochloride. The crosslinkable functional groups can be present at any point along the polymer chain of the present composition so long as their location does not interfere with the intended function thereof. Furthermore, the crosslinkable functional groups can be present in the polymer chain of the present invention in numbers greater than two, so long as the intended function of the present composition is not compromised. An example of a coating having the above-recited characteristics is found in, for example, Loomis, U.S. Pat. No. 5,854,382. This coating is exemplary of the types of coatings that can be used in the invention.

Also contemplated by the present invention is the use of carriers, coatings, matrices or grafts that are capable of promoting the release of an RNAi agent from the coating. For example, in some embodiments, the RNAi agent is dispersed throughout a hydrogel. As the hydrogel degrades by hydrolysis or enzymatic action, the RNAi agent is released. Alternatively, the coating may promote the release of a biologically active material by forming pores once the resulting article is placed in a particular environment (e.g., in vivo). In one embodiment, the pores communicate with a reservoir containing the RNAi agent. Other such coating components that promote the release of an RNAi agent from materials are known to those of skill in the art.

Microencapsulation of RNAi Agents

In another embodiment, the RNAi agent or agents are incorporated into a polymeric component by encapsulation in a microcapsule. The microcapsule is preferably fabricated from a material different from that of the bulk of the carrier, coating, matrix or graft. Preferred microcapsules are those which are fabricated from a material that undergoes erosion in the host, or those which are fabricated such that they allow the RNAi agent to diffuse out of the microcapsule. Such microcapsules can be used to provide for the controlled release of the encapsulated RNAi agent from the microcapsules.

Numerous methods are known for preparing microparticles of any particular size range. In the various delivery vehicles of the present invention, the microparticle sizes may range from about 0.2 μm up to about 100 μm. Synthetic methods for gel microparticles, or for micro particles from molten materials are known, and include polymerization in emulsion, in sprayed drops, and in separated phases. For solid materials or preformed gels, known methods include wet or dry milling or grinding, pulverization, size separation by air jet, sieve, and the like.

Microparticles can be fabricated from different polymers using a variety of different methods known to those skilled in the art. Exemplary methods include those set forth below detailing the preparation of polylactic acid and other microparticles. Polylactic acid microparticles are preferably fabricated using one of three methods: solvent evaporation, as described by Mathlowitz, et al, J. Scanning Microscopy 4:329 (1990); Beck, et al, Fertil Steril 31: 545 (1979); and Benita, et al, J. Pharm. Sci. 73: 1721 (1984); hot-melt microencapsulation, as described by Mathiowitz, et al, Reactive Polymers 6: 275 (1987); and spray drying. Exemplary methods for preparing microencapsulated bioactive materials useful in practicing the present invention are set forth below.

In the solvent evaporation method, the microcapsule polymer is dissolved in a volatile organic solvent, such as methylene chloride. The RNAi agent (either soluble or dispersed as fine particles) is added to the solution, and the mixture is suspended in an aqueous solution that contains a surface active agent such as poly(vinyl alcohol). The resulting emulsion is stirred until most of the organic solvent has evaporated, leaving solid microparticles. The solution is loaded with the RNAi agent and suspended in vigorously stirred distilled water containing poly(vinyl alcohol) (Sigma). After a period of stirring, the organic solvent evaporates from the polymer, and the resulting microparticles are washed with water and dried overnight in a lyophilizer. Microparticles with different sizes (1-1000 μm) and morphologies can be obtained by this method. This method is useful for relatively stable polymers like polyesters and polystyrene. Labile polymers such as polyanhydrides, may degrade during the fabrication process due to the presence of water. For these polymers, the following two methods, which are performed in completely anhydrous organic solvents, are preferably used.

In the hot melt encapsulation method, the polymer is first melted and then mixed with the solid particles of biologically active material that have preferably been sieved to less than 50 microns. The mixture is suspended in a non-miscible solvent (like silicon oil) and, with continuous stirring, heated to about 5° C. above the melting point of the polymer. Once the emulsion is stabilized, it is cooled until the polymer particles solidify. The resulting microparticles are washed by decantation with a solvent such as petroleum ether to give a free-flowing powder. Microparticles with sizes ranging from about 1 to about 1000 microns are obtained with this method. The external surfaces of capsules prepared with this technique are usually smooth and dense. This procedure is preferably used to prepare microparticles made of polyesters and polyanhydrides.

The solvent removal technique is preferred for polyanhydrides. In this method, the RNAi agent is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. Microparticles that range from about 1 to about 300 μm can be obtained by this procedure. The external morphology of spheres produced with this technique is highly dependent on the type of polymer spray drying, the polymer is dissolved in methylene chloride. A known amount of the RNAi agent is suspended or co-dissolved in the polymer solution. The solution or the dispersion is then spray-dried. Microparticles ranging between about 1 to about 10 μm are obtained with a morphology which depends on the type of polymer used.

In one embodiment, the RNAi agent is encapsulated in microcapsules that comprise a sodium alginate envelope. Microparticles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath for about twenty to thirty minutes in order to allow sufficient time for gelation to occur. Microparticle size is controlled by using various size extruders or varying either the nitrogen gas or polymer solution flow rates.

Liposomes can aid in the delivery of the RNA agents (whether siRNAi agents or ddRNAi agents) to a particular tissue and also can increase the half-life of the RNA agent. Liposomes are commercially available from a variety of suppliers. Alternatively, liposomes can be prepared according to methods known to those skilled in the art, for example, as described in Eppstein, et al, U.S. Pat. No. 4,522,811. In general, liposomes are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka, et al. (1980), Ann. Rev. Biophys. Bioeng. 9: 467; and U.S. Pat. Nos. 4,235,871, 4,501,728, 4,837,028, and 5,019,369. In one embodiment, the liposomes encapsulating the RNAi agent according to the present invention comprise a ligand molecule that can target the liposome to a particular cell or tissue at or near the site of vascular injury. Ligands which bind to receptors prevalent in vascular tissue, such as monoclonal antibodies that bind to vascular tissue.

In one embodiment, the liposomes encapsulating the RNAi agents of the present invention are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand. Opsonization-inhibiting moieties for use in preparing the liposomes in one embodiment of the present invention are large hydrophilic polymers that are bound to the liposome membrane. As used herein, an opsonization inhibiting moiety is “bound” to a liposame membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016. Liposomes modified with opsonization-inhibition moieties thus remain in the circulation much longer than unmodified liposomes. For this reason, such liposomes are sometimes called “stealth” liposomes. Stealth liposomes are known to accumulate in tissues fed by porous or “leaky” microvasculature. In addition, the reduced uptake by the RES lowers the toxicity of stealth liposomes by preventing significant accumulation in the liver and spleen.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; laminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.” The opsonization inhibiting moiety can be bound to the liposome membrane by anyone of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH3 and a solvent mixture such as tetrahydrofuran and water in a 30:12 ratio at 60° C.

The above-recited microparticles and liposomes and methods of preparing microparticles and liposomes are offered by way of example and are not intended to define the scope of microparticles or liposomes of use in the present invention. It will be apparent to those of skill in the art that an array of microparticles or liposomes, fabricated by different methods, are of use in the present invention.

In another embodiment of the present invention, the methods of the invention include the use of two or more populations of RNAi agents. The populations are distinguished by, for example, sequence, or by having different rates of release from a carrier, coating, matrix or graft of the invention. Two or more different rates of release can be obtained by, for example, incorporating one RNAi agent population into the bulk coating and another RNAi agent population into microcapsules embedded in the bulk coating. In another exemplary embodiment, the two RNAi or more agents are encapsulated in microspheres having distinct release properties. For example, a first agent is encapsulated in a liposome and a second RNAi agent is encapsulated in an alginate microsphere.

Other characteristics of the RNAi agent populations in addition to their release rates can be varied as well. For example, the two RNAi agent populations can consist of agents having the same or different sequences, and the sequences can target different portions of the same gene, or portions of different genes. Also, one or more RNAi agents can be delivered as siRNAs, and other RNAi agents can be delivered as ddRNAs. The concentrations of the two or more RNAi populations can differ from one another. For example, in certain applications it is desirable to have one agent released rapidly (e.g., an RNAi agent targeting a gene involved in blood clotting) at a first concentration, while a second RNAi agent is released more slowly at a second concentration (e.g., an inhibitor of tissue overgrowth). Furthermore when two or more distinct RNAi agents are used they can be distributed at two or more unique sites within the delivery vehicle.

In certain embodiments, a solid, flexible therapeutic is formed by dispensing a flowable polymer, or polymer precursor, formulation onto the surface of a matrix or graft. The formulation can be applied by any convenient technique. For example, the formulation can be applied by brushing, spraying, extruding, dripping, injecting, or painting. Spraying, via aerosolization is a preferred method of administration because it minimizes the amount of formulation applied to the site of insult while maximizing uniformity. A thin, substantially uniform matrix, such as that formed by spraying, can also be called a film. Typically, the film has a thickness of about 10 μm to about 10 mm, more preferably from about 20 μm to about 5 mm. Spraying is a preferred method for applying the polymer formulation to a large surface area. In contrast, dripping may be preferred for applying the polymer formulation to a small surface area.

Characterization of the RNAi agent, the carriers, coating, matrices and stents or stent grafts and the combination thereof can be performed at different loadings of RNAi agent to investigate nucleic acid formulation, and carrier, coating, matrix and stent or stent graft formulation and encapsulation properties and. morphological characteristics. Microparticle size can be measured by quasi-elastic light scattering (QELS), size-exclusion, chromatography (SEC) and the like. Drug loading can be measured by dissolving the coating or the microparticles into an appropriate solvent and assaying the amount of biologically active molecules using one or more art-recognized techniques. Useful techniques include, for example, spectroscopy (e.g., IR, NMR, UV/Vis, fluorescence, etc.), mass spectrometry, elemental analysis, HPLC, HPLC coupled with one or more spectroscopic modalities, and other appropriate means.

Patients can be diagnosed for restenosis using known methods, such as X-ray fluoroscopic examination of dye flowing through a particular region of a blood vessel or other visual techniques, the presence of symptoms such as pain, based on clinical judgment, or signs evidenced physical examination. Alternatively, it can be assumed that restenosis will arise due to performance of procedures such as angioplasty, arterial bypass graft, peripheral bypass surgery, or organ transplantation and the patient treated based on the assumption that injury or disease will inevitably arise. In one embodiment, a carrier, coating, matrix, stent or stent graft comprising an RNAi agent is applied to the site of insult during an open-field procedure. In another embodiment, the therapeutic device is placed at the site of insult via percutaneous means. In yet another embodiment, one or more RNAi agents is applied to a stent graft or stent and the stent graft or stent is deployed in the vessel to be treated.

If restenosis had been observed prior to deployment of the stent or stent graft or delivery of the matrix or flowable carrier, the regression of restenosis is typically evidenced by a decrease in pain or other symptoms of decreased blood flow, or through the use of imaging techniques. The decrease in restenosis or increase in flow rate through the injured vessel can be monitored by the same methods used to initially diagnose the injury to the vascular endothelium or blockage of the blood vessel.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material or process to the objective, spirit and scope of the present invention. All such modifications are intended to be within the scope of the invention.

All references cited herein are to aid in the understanding of the invention, and are incorporated in their entireties for all purposes without limitation.

### BIBLIOGRAPHY

Animal Cell Culture (R.t. Freshney, ed. 1986);
Baim et al., Am. J. Cardiol. 71:364-366 (1993);
Beck, et al., Fertil. Steril 31: 545 (1979);
Benita, et al., J. Pharm. Sci. 73: 1721 (1984);
Boden, et, al, J. ViroL 77(21): 115231-35 (2003);
Bowerman et al., Cathet. Cardiovasc. Diagn. 24:248-251 (1991);
Carlin et al., Am J Physiol Lung Cell Mol Physiol. 284(6):L 1020-6 (2003);
Casscells et al., Proc Natl Acad Sci USA. 89 (15): 7159-7163 (1992);
Chen, et al., Molecular Therapy. 8(3):495-500 (2003);
Chervu et al., Surg. Gynecol. Obstet. 171:433-447, 1990);
Cipollone et al., Circulation 108(22): 2776-82 (2003);
Cohn et al., J. Biomed. Mater. Res. 22: 993-1009 (1988);
Coombes et al., J Infect Dis. 185(11): 1621-30 (2002);
De Cristofaro and De Candia, J Thromb Thrombolysis. 15(3): 151-63 (2003);
DNA Cloning: A Practical Approach, Volumes I and II (D. N. Glover, ed. 1985);
Duan, Mol Cell Endocrinol. 206(1-2): 75-83 (2003);
Elbashir, et al., Nature, 411:494-498 (2001);
Everse, Vox Sang. 83 Suppl1:375-82 (2002);
Fang et al., Arterioscler Thromb Vasc Biol. 24(4): 787-92 (2004).
Ferrero et al., Arterioscler Thromb Vasc Biol. 23(12): 2223-8 (2003);
Fire, et al., Nature, 391:806-11 (1998);
Ghannem et al., Ann. Cardiol. Angeiol. 45:287-290 (1996);
Gordon et al., J. Am. Coll. Cardiol. 21:1166-1174;
Grimm et al. Blood. 2003-02-0495;
Grzeszkiewicz et al., Endocrinology 143(4): 1441-50 (2002);
Harborth, et al., Antisense Nucleic Acid Drug Rev. 13(2): 83-105 (2003);
Heine et al., Kidney Int. 64(3): 1101-7 (2003);
Higashibata, et al., J. Bone Miner. Res. January 19(1):78-88 (2004);
Hoggatt, et al., Circ. Res., December 91(12):1151-59 (2002);
Hokimoto et al., Circ J. 66(1): 114-6 (2002);
Holmes and Schwartz, Composites Science and Technology, 38: 1-21 (1990);
Hughes et al. Cardiovasc Res. 27: 1214-1219 (1993);
Humphries et al., Eur Heart J. 23(9): 721-5 (2002);
Isoda et al., Circ Res. 91(1): 77-82 (2002);
Kay, et al., Nature. 424: 251 (2003);
Khanolkar, Indian Heart J. 48:281-282 (1996);
Kurreck, Eur. J. Bioch. 270:1628-44 (2003);
Kuzuya et al., Circulation 108(11): 1375-81 (2003);
LaRochelle et al., Cancer Res. 62(9): 2468-73 (2002);
Li et al., Circulation 106: 854 (2002);
Maas-Szabowski et al., J Cell Sci. 116: 2937-48 2003);
Macander et al, Cathet. Cardiovasc. Oiagn. 32:125-131;
Maraia, et al., Nucleic Acids Res 22(15):3045-52 (1994);
Maraia, et al., Nucleic Acids Res 24(18):3552-59 (1994);
Maniatis, Fritsch & Sambrook, Molecular Cloning: A Laboratory Manual (1982); Marculescu et al., Thromb Haemost. 90(3): 491-500 (2003);
Mathiowitz, et al., J. Scanning Microscopy 4:329 (1990);
Mathiowitz, et al., Reactive Polymers 6: 275 (1987);
Matsumoto et al., Arterioscler Thromb Vasc Biol. 24(1): 181-6 (2004);
Meier, Eur. Heart J. 10 (suppl G):64-68 (1989);
Mingozzi, et al., J. Virol 76(20): 10497-502 (2002);
Moris et al., Am. Heart. J. 131:834-836 (1996);
Mosesson, J Thromb Haemost. 1(2): 231-8 (2003);
Nucleic Acid Hybridization (B. D. Hames & S. J. Higgins, eds. (1984);
Oligonucleotide Synthesis (M. J. Gait, ed. 1984);
Pastore et al., Circulation Research. 77: 519-529 (1995);
Perri, et ai, J. Virol 74(20):9802-07 (2002);
Protective Groups in Organic Chemistry, McOmie, ed., Plenum Press, NY, N.Y. (1973);
Protective Groups in Organic Synthesis, Greene, ed., John Wiley & Sons, NY (1981);
Ragosta et al., Circulation 89: 11262-127 (1994);
RNA Viruses: A practical Approach, (Alan, J. Cann, Ed., Oxford University Press, (2000);
Sajid and Stouffer, Thromb Haemost. 87(2): 187-93 (2002);
Sambrook et al., Molecular Cloning: A Laboratory Manual, 2nd Ed., Cold Spring Harbor Press, Cold Spring Harbor, N.Y. (1989);
Sawhney et al., Macromolecules 26: 581-587 (1993);
Schafer, Tex Heart Inst J. 24(2): 90-96 (1997);
Scharf et al., Blut 55:1131-1144 (1987);
Schomig et al., J. Am. Coll. Cardiol. 23:1053-1060 (1994);
Schwartz, et al., N. Engl. J. Med. 318:1714-1719, (1988);
Sohal, et al., Circ. Res. July 89(1):20-25 (2001);
Strauss et al., J. Am. Coll. CardioL 20:1465-1473 (1992);
Szoka et al., Ann. Rev. Biophys. Bioeng. 9: 467 (1980);
Taniguchi et al., Jpn Circ J. 65(10): 897-900 (2001);
Thomas, et al., Nature Reviews. Genetics 4:346-58 (2003);
Thomas, et al., J. Virol in press
Tomar et al., Oncogene. 22: 5712-15 (2003);
Tuschl, et al., Genes and Dev., 13:3191-97 (1999);
U.S. Pat. No. 3,826,678;
U.S. Pat. No. 4,235,871;
U.S. Pat. No. 4,438,253;
U.S. Pat. No. 4,501,728;
U.S. Pat. No. 4,521,564;
U.S. Pat. No. 4,522,811;
U.S. Pat. No. 4,826,945;
U.S. Pat. No. 4,837,028;
U.S. Pat. No. 4,842,575;
U.S. Pat. No. 4,916,193;
U.S. Pat. No. 4,920,016;
U.S. Pat. No. 4,994,071;
U.S. Pat. No. 5,019,369;
U.S. Pat. No. 5,034,265;
U.S. Pat. No. 5,053,048;
U.S. Pat. No. 5,092,877;
U.S. Pat. No. 5,163,952;
U.S. Pat. No. 5,202,413;
U.S. Pat. No. 5,304,121;
U.S. Pat. No. 5,344,455;
U.S. Pat. No. 5,410,016;
U.S. Pat. No. 5,527,532;
U.S. Pat. No. 5,529,914;
U.S. Pat. No. 5,558,642;
U.S. Pat. No. 5,739,288;
U.S. Pat. No. 5,770,194;
U.S. Pat. No. 5,837,313;
U.S. Pat. No. 5,854,382;
U.S. Pat. No. 5,879,697;
U.S. Pat. No. 5,880,131;
U.S. Pat. No. 5,900,245;
U.S. Pat. No. 5,900,246;
U.S. Pat. No. 6,218,181;
U.S. Pat. No. 6,573,099;
U.S. Pat. No. 6,673,611;
U.S. Pat. No. 6,730,313;
U.S. Publ. No. 2002/0162126
U.S. Publ. No. 2004/0147023;
U.S. Publ. No. 2004/0147022;
U.S. Publ. No. 2004/0147470;
U.S. Publ. No. 2004/0161777;
U.S. Publ. No. 2004/0161844;
U.S. Publ. No. 2004/0171030;
U.S. Publ. No. 2004/0171031;

What is claimed is:

1. A method for preventing or reducing restenosis, said method comprising delivering an RNAi agent in or adjacent to vascular tissue, wherein the RNAi agent targets a gene involved in restenosis.

2. The method of claim 1, wherein the RNAi agent comprises a nucleotide sequence that is at least 70% identical to a region of the targeted gene.

3. The method of claim 1, wherein the gene is involved in blood clotting or smooth muscle or endothelial proliferation.

4. The method of claim 1, wherein the gene is selected from the group consisting of platelet derived growth factor, epidermal growth factor, transforming growth factor-α, fibroblast growth factor, transforming growth factor-β, insulin-like growth factor-I, fibrin, thrombin, and platelet activating factor.

5. The method of claim 1, wherein delivery of the RNAi agent comprises delivering the RNAi agent with a carrier, coating, or delivery vehicle.

6. The method of claim 5, wherein the RNAi agent is delivered with a delivery vehicle, and the delivery vehicle is selected from the group consisting of a stent, stent graft, drug delivery matrix, or a flowable liquid or a semi-liquid material.

7. The method of claim 1, wherein the RNAi agent is reversibly associated with a carrier, coating, or matrix.

8. The method of claim 1, wherein the RNAi agent is covalently attached to a carrier or coating.

9. The method of claim 1, wherein the RNAi agent is delivered in or adjacent the vascular tissue by a viral delivery system.

10. A therapeutic device comprising an RNAi agent that targets a gene involved in restenosis and a carrier, coating, or delivery vehicle.
